(19) United States
(12) Patent Application Publication
(10) Pub. No.: US 2014/0275281 A1
(43) Pub. Date: Sep. 18, 2014

(54) Geranylgeranyl acetone and derivatives thereof for intranasal administration

(71) Applicant: COYOTE PHARMACEUTICALS, INC., MENLO PARK, CA (US)

(72) Inventors: Hiroaki Serizawa, Menlo Park, CA (US); Tilmann M. Brotz, Menlo Park, CA (US)

(73) Assignee: COYOTE PHARMACEUTICALS, INC., MENLO PARK, CA (US)

(21) Appl. No.: 13/815,741

(22) Filed: Mar. 15, 2013

(51) Int. Cl. A61K 31/121

(52) U.S. Cl. CPC A61K 31/121
USPC 514/675

(57) ABSTRACT

Provide herein are intranasal compositions which include geranylgeranyl acetone (GGA) and/or derivatives thereof and methods for treating a neural disease, disorder or condition with the same.

### FIELD OF THE INVENTION

This invention provides therapeutic compositions suitable for intranasal administration, which include geranylgeranyl acetone (GGA) and/or derivatives thereof. This invention also provides therapeutic methods for treating a neural disease, disorder or condition by the intranasal administration of compositions that include geranylgeranyl acetone (GGA) and derivatives thereof. Preferably, GGA or the GGA derivative is enriched in the all trans isomer, compared to the relative amount of the trans isomer in the mixtures of cis and trans isomers of GGA or the GGA derivative.

### STATE OF THE ART

Geranylgeranyl acetone (GGA) has the formula:

[chemical structure]

and is reported to have neuroprotective and related effects. See, for example, PCT Pat. App. Pub. No. WO 2012/031028 and PCT Pat. App. No. PCT/US2012/027147, each of which is incorporated herein by reference in its entirety.

### SUMMARY OF THE INVENTION

In one aspect of the invention, an intranasal composition is provided, the composition comprising an effective amount of geranylgeranyl acetone (GGA) or a GGA derivative, or a pharmaceutically acceptable salt thereof, and a pharmaceutically acceptable excipient.

Preferably, the GGA or the GGA derivative includes the all-trans (hereinafter “trans”) form or substantially the trans form of the GGA or the GGA derivative. As used herein, “substantially” in the context of cis/trans configurations refers to at least 80%, more preferably at least 90%, yet more preferably at least 95%, and most preferably at least 99% of the desired configuration, which can include at least 80%, more preferably at least 90%, yet more preferably at least 95%, and most preferably at least 99% of the trans isomer. In certain preferred embodiments of the invention, the GGA or a GGA derivative exists at least 80%, or at least 90%, or at least 95%, or at least 99% in the trans isomer.

In certain aspects, this invention relates to pharmaceutical uses of geranylgeranyl acetone (GGA) and GGA derivatives, pharmaceutical compositions of isomers of geranylgeranyl acetone, preferably synthetic geranylgeranyl acetone, and GGA derivatives, and methods of using such compounds and pharmaceutical compositions. In certain aspects, this invention relates to a 5-trans isomer compound of formula VI:

[chemical structure]

wherein VI is at least 80% in the 5E, 9E, 13E configuration. In one embodiment, this invention utilizes a compound, which is synthetic 5E, 9E, 13E geranylgeranyl acetone. In another embodiment, the synthetic 5E, 9E, 13E geranylgeranyl acetone is free of 5Z, 9E, 13E geranylgeranyl acetone. In another aspect, this invention provides a pharmaceutical composition comprising synthetic GGA or synthetic 5E, 9E, 13E GGA, and at least one pharmaceutical excipient.

Another aspect of this invention relates to a synthetic 5-cis isomer compound of formula VII:

[chemical structure]

wherein VII is at least 80% in the 5Z, 9E, 13E configuration, or a ketal thereof of formula XII:

[chemical structure]

wherein each R₇₀ independently is C₁-C₆ alkyl, or two R₇₀ groups together with the oxygen atoms they are attached to form a 5 or 6 membered ring, which ring is optionally substituted with 1-3, preferably 1-2, C₁-C₆ alkyl groups. Preferably, the two R₇₀ groups are the same. In one embodiment, R₇₀ is, methyl, ethyl, or propyl. In another embodiment, the cyclic ring is:

[chemical structure]

In another aspect provide herein are compounds wherein GGA or a derivative thereof is conjugated to an anti-cancer agent. In one embodiment, the conjugate is of formula:

[chemical structure]

wherein R¹-R⁵, m, and n are defined as in Formula (II) herein, L¹⁰ is a bond or a linker joining the isoprenyl portion to the Drug, and the Drug is preferably an antibiotic or a glaucoma drug, or is an anticancer agent, or is an antiviral agent. In certain preferred embodiments, the linker is a bond, methylene, or carbonyl. In certain other preferred embodiments, the linker joins the isoprenyl portion to a carbonyl moiety, or an oxygen, nitrogen, or sulfur atom of the drug. In yet another preferred embodiment, R¹-R⁵ are methyl, and m and n are 1. Such conjugates are formulated and administered in accordance with this invention.

As to intranasal delivery, the surface area of the nostril is small and thus can absorb only a limited volume of any intranasal composition. As such, the concentration of the GGA or the GGA derivative in the intranasal composition is contemplated to be sufficiently high e.g., 0.1-20% (weight/volume) to compensate for the small volumes, e.g., 0.01-2 mL, of the intranasal composition that are administered to each nostril. In certain embodiments, the composition includes 0.1-5%, or preferably 5-10%, or more preferably 10-15% or 15-20% (weight/volume) of GGA or a GGA derivative, or a pharmaceutically acceptable salt thereof.

The intranasal compositions described herein are contemplated to be administered to each or either nostril one or more times, e.g., 1, 2, 3, 4, 5, 6, 7 or 8 times per day. It is further contemplated that a sufficient time delay, e.g., of 1-30 minutes or more, such as time delays of 30 minutes, 1, 2, 3, 4, 8, 12, 24 or 48 hours may be used between each administration. Without being bound by theory, it is believed that each nostril can absorb only a limited volume of any intranasal composition and thus it quickly becomes saturated by the intranasal compositions described herein.

It is contemplated that an effective amount of GGA or a derivative thereof is efficiently administered by employing the intranasal compositions described herein. In some embodiments the intranasal formulation of GGA or a derivative contains between 1-55, 5-50, 10-40, or 20-30 mg/kg/day.

In certain embodiments, the composition is in the form of a solution or suspension. In other embodiments, said excipient comprises a bioadhesive and/or an intranasal absorption promoter. Said intranasal absorption promoter, in some embodiments, is one or more of a chelating agent, POE (9) lauryl alcohol, sodium glycocholate and lysophosphatidyl choline.

In another aspect of the invention, a method is provided for administering intranasally an effective amount of the compositions to a subject in need thereof. As used herein, subject or patient refers to a mammal, particularly preferably humans.

In another aspect of the invention, a method is provided for treating a neural disease, disorder or condition and/or reducing one or more negative effects of a neural disease, disorder or condition comprising administering intranasally an effective amount of any of the compositions described herein to a subject in need thereof.

According to another aspect of this invention, a method is provided for delivering a GGA derivative to the brain and/or the spinal chord of a patient, which method comprises administering an intranas composition intranasally to said patient in an amount sufficient to introduce an effective amount of GGA derivative into the brain and/or the spinal chord. As used herein, an effective amount refers to a therapeutically effective amount or to a an amount effectively measured in the brain and/or the spinal chord.

Some embodiments provided herein describe a method for inducing expression of a heat shock protein in a subject in need thereof, the method comprising administering to the subject a therapeutically effective amount of GGA or a GGA derivative thereof, wherein the GGA or GGA derivative thereof is administered intranasally to said subject.

Other embodiments provided herein describe a method for inhibiting neural death in a subject in need thereof, the method comprising administering to the subject a therapeutically effective amount of GGA or a GGA derivative thereof, wherein the GGA or GGA derivative thereof is administered intranasally to said subject.

In yet other embodiments, various bacterial and viral disorders, and cancers of the eye, the brain, and the spinal chord, and nerves, including without limitation, nerves in the brain, eye, and the spinal chord are treated in accordance with this invention. In some embodiments, the disorder is glaucoma. In another embodiment, the disorder is herpes.

### DETAILED DESCRIPTION OF THE INVENTION

### Definitions

It must be noted that as used herein and in the appended claims, the singular forms “a”, “an”, and “the” include plural referents unless the context clearly dictates otherwise. Thus, for example, reference to “a solvent” includes a plurality of such solvents.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition or process consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations. Each numerical parameter should at least be construed in light of the number of reported significant digits and by applying ordinary rounding techniques.

As used herein, Cₘ-Cₙ, such as C₁-C₁₀, C₁-C₆, or C₁-C₄ when used before a group refers to that group containing m to n carbon atoms.

The term “about” when used before a numerical designation, e.g., temperature, time, amount, and concentration, including range, indicates approximations which may vary by (+) or (−) 10%, 5% or 1%.

The term “alkoxy” refers to —O-alkyl.

The term “nitro” refers to —NO₂.

The term “alkyl” refers to monovalent saturated aliphatic hydrocarbyl groups having from 1 to 10 carbon atoms (i.e., C₁-C₁₀ alkyl) or 1 to 6 carbon atoms (i.e., C₁-C₆ alkyl), or 1 to 4 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl RCH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “alkenyl” refers to monovalent aliphatic hydrocarbyl groups having from 2 to 25 carbon atoms or 2 to 6 carbon atoms and 1 or more, preferably 1, carbon carbon double bond. Examples of alkenyl include vinyl, allyl, dimethyl allyl, and the like.

The term “alkynyl” refers to monovalent aliphatic hydrocarbyl groups having from 2 to 10 carbon atoms or 2 to 6 carbon atoms and 1 or more, preferably 1, carbon carbon triple bond —(C≡C)—. Examples of alkynyl include ethynyl, propargyl, dimethylpropargyl, and the like.

The term “acyl” refers to —C(O)-alkyl, where alkyl is as defined above.

The term “aryl” refers to a monovalent, aromatic mono- or bicyclic ring having 6-10 ring carbon atoms. Examples of aryl include phenyl and naphthyl. The condensed ring may or may not be aromatic provided that the point of attachment is at an aromatic carbon atom. For example, and without limitation, the following is an aryl group:

[chemical structure]

The term “—CO₂H ester” refers to an ester formed between the —CO₂H group and an alcohol, preferably an aliphatic alcohol. A preferred example included —CO₂R^{E}, wherein R^{E} is alkyl or aryl group optionally substituted with an amino group.

The term “chiral moiety” refers to a moiety that is chiral. Such a moiety can possess one or more asymmetric centers. Preferably, the chiral moiety is enantiomerically enriched, and more preferably a single enantiomer. Non limiting examples of chiral moieties include chiral carboxylic acids, chiral amines, chiral amino acids, such as the naturally occurring amino acids, chiral alcohols including chiral steroids, and the likes.

The term “cycloalkyl” refers to a monovalent, preferably saturated, hydrocarbyl mono-, bi-, or tricyclic ring having 3-12 ring carbon atoms. While cycloalkyl, refers preferably to saturated hydrocarbyl rings, as used herein, it also includes rings containing 1-2 carbon-carbon double bonds. Nonlimiting examples of cycloalkyl include cyclopropyl, cyclobutyl, cyclopentyl, cyclohexyl, cycloheptyl, adamentyl, and the like. The condensed rings may or may not be non-aromatic hydrocarbyl rings provided that the point of attachment is at a cycloalkyl carbon atom. For example, and without limitation, the following is a cycloalkyl group:

[chemical structure]

The term “halo” refers to F, Cl, Br, and/or I.

The term “heteroaryl” refers to a monovalent, aromatic mono-, bi-, or tricyclic ring having 2-14 ring carbon atoms and 1-6 ring heteroatoms selected preferably from N, O, S, and P and oxidized forms of N, S, and P, provided that the ring contains at least 5 ring atoms. Nonlimiting examples of heteroaryl include furan, imidazole, oxadiazole, oxazole, pyridine, quinoline, and the like. The condensed rings may or may not be a heteroatom containing aromatic ring provided that the point of attachment is a heteroaryl atom. For example, and without limitation, the following is a heteroaryl group:

[chemical structure]

The term “heterocyclyl” or heterocycle refers to a non-aromatic, mono-, bi-, or tricyclic ring containing 2-10 ring carbon atoms and 1-6 ring heteroatoms selected preferably from N, O, S, and P and oxidized forms of N, S, and P, provided that the ring contains at least 3 ring atoms. While heterocyclyl preferably refers to saturated ring systems, it also includes ring systems containing 1-3 double bonds, provided that they ring is non-aromatic. Nonlimiting examples of heterocyclyl include, azalactones, oxazoline, piperidinyl, piperazinyl, pyrrolidinyl, tetrahydrofuranyl, and tetrahydropyranyl. The condensed rings may or may not contain a non-aromatic heteroatom containing ring provided that the point of attachment is a heterocyclyl group. For example, and without limitation, the following is a heterocyclyl group:

[chemical structure]

The term “hydrolyzing” refers to breaking an R^{H}—O—CO—, R^{H}—O—CS—, or an R^{H}—O—SO₂-moiety to an R^{H}—OH, preferably by adding water across the broken bond. A hydrolyzing is performed using various methods well known to the skilled artisan, non limiting examples of which include acidic and basic hydrolysis.

The term “oxo” refers to a C═O group, and to a substitution of 2 geminal hydrogen atoms with a C═O group.

The term “pharmaceutically acceptable” refers to safe and non-toxic for in vivo, preferably, human administration.

The term “pharmaceutically acceptable salt” refers to a salt that is pharmaceutically acceptable.

The term “salt” refers to an ionic compound formed between an acid and a base. When the compound provided herein contains an acidic functionality, such salts include, without limitation, alkai metal, alkaline earth metal, and ammonium salts. As used herein, ammonium salts include, salts containing protonated nitrogen bases and alkylated nitrogen bases. Exemplary, and non-limiting cations useful in pharmaceutically acceptable salts include Na, K, Rb, Cs, NH₄, Ca, Ba, imidazolium, and ammonium cations based on naturally occurring amino acids. When the compounds utilized herein contain basic functionally, such salts include, without limitation, salts of organic acids, such as caroboxylic acids and sulfonic acids, and mineral acids, such as hydrogen halides, sulfuric acid, phosphoric acid, and the likes. Exemplary and non-limiting anions useful in pharmaceutically acceptable salts include oxalate, maleate, acetate, propionate, succinate, tartrate, chloride, sulfate, bisalfate, mono-, di-, and tribasic phosphate, mesylate, tosylate, and the likes.

The term “substantially pure trans isomer” refers to a trans isomer that is by molar amount 95%, preferably 96%, more preferably 99%, and still more preferably 99.5% or more a trans isomer with the rest being the corresponding cis isomer.

“Trans” in the context of GGA and GGA derivatives refer to the GGA scaffold as illustrated below:

[chemical structure]

wherein R¹-R⁵ is defined herein and q is 0-2. As shown, each double bond is in a trans or E configuration. In contrast, a cis form of GGA or a GGA derivative will contain one or more of these bonds in a cis or Z configuration.

The term “neuroprotective” refers to reduced toxicity of neurons as measured in vitro in assays where neurons susceptible to degradation are protected against degradation as compared to control. Neuroprotective effects may also be evaluated in vivo by counting neurons in histology sections.

The term “neuron” or “neurons” refers to all electrically excitable cells that make up the central and peripheral nervous system. The neurons may be cells within the body of an animal or cells cultured outside the body of an animal. The term “neuron” or “neurons” also refers to established or primary tissue culture cell lines that are derived from neural cells from a mammal or tissue culture cell lines that are made to differentiate into neurons. “Neuron” or “neurons” also refers to any of the above types of cells that have also been modified to express a particular protein either extrachromosomally or intrachromosomally. “Neuron” or “neurons” also refers to transformed neurons such as neuroblastoma cells and support cells within the brain such as glia.

The term “protein aggregates” refers to a collection of proteins that may be partially or entirely mis-folded. The protein aggregates may be soluble or insoluble and may be inside the cell or outside the cell in the space between cells. Protein aggregates inside the cell can be intranuclear in which they are inside the nucleus or cytoplasm in which they are in the space outside of the nucleus but still within the cell membrane. The protein aggregates described in this invention are granular protein aggregates.

As used herein, the term “protein aggregate inhibiting amount” refers to an amount of compound that inhibits the formation of protein aggregates at least partially or entirely. Unless specified, the inhibition could be directed to protein aggregates inside the cell or outside the cell.

As used herein, the term “intranuclear” or “intranuclearly” refers to the space inside the nuclear compartment of an animal cell.

The term “cytoplasm” refers to the space outside of the nucleus but within the outer cell wall of an animal cell.

As used herein, the term “pathogenic protein aggregate” refers to protein aggregates that are associated with disease conditions. These disease conditions include but are not limited to the death of a cell or the partial or complete loss of the neuronal signaling among two or more cells. Pathogenic protein aggregates can be located inside of a cell, for example, pathogenic intracellular protein aggregates or outside of a cell, for example, pathogenic extracellular protein aggregates.

As used herein, the term “SBMA” refers to the disease spinal and bulbar muscular atrophy. Spinal and bulbar muscular atrophy is a disease caused by pathogenic androgen receptor protein accumulation intranuclearly.

As used herein, the term “ALS” refers to amyotrophic lateral sclerosis disease.

As used herein, the term “AD” refers to Alzheimer's disease.

The term “neurotransmitter” refers to chemicals which transmit signals from a neuron to a target cell. Examples of neurotransmitters include but are not limited to: amino acids such as glutamate, aspartate, serine, γ-aminobutyric acid, and glycine; monoamines such as dopamine, norepinephrine, epinephrine, histamine, serotonin, and melatonin; and other molecules such as acetycholine, adenosine, anadamide, and nitric oxide.

The term “synapse” refers to junctions between neurons. These junctions allow for the passage of chemical signals from one cell to another.

The term “G protein” refers to a family of proteins involved in transmitting chemical signals outside the cell and causing changes inside of the cell. The Rho family of G proteins is small G protein, which are involved in regulating actin cytoskeletal dynamics, cell movement, motility, transcription, cell survival, and cell growth. RHOA, RAC1, and CDC42 are the most studied proteins of the Rho family. Active G proteins are localized to the cellular membrane where they exert their maximal biological effectiveness.

The terms “treat”, “treating” or “treatment”, as used herein, include alleviating, abating or ameliorating a disease or condition or one or more symptoms thereof, preventing additional symptoms, ameliorating or preventing the underlying metabolic causes of symptoms, inhibiting the disease or condition, e.g., arresting or suppressing the development of the disease or condition, relieving the disease or condition, causing regression of the disease or condition, relieving a condition caused by the disease or condition, or suppressing the symptoms of the disease or condition, and are intended to include prophylaxis. The terms also include relieving the disease or conditions, e.g., causing the regression of clinical symptoms. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the individual, notwithstanding that the individual is still be afflicted with the underlying disorder. For prophylactic benefit, the compositions are administered to an individual at risk of developing a particular disease, or to an individual reporting one or more of the physiological symptoms of a disease, even though a diagnosis of this disease has not been made.

The terms “preventing” or “prevention” refer to a reduction in risk of acquiring a disease or disorder (i.e., causing at least one of the clinical symptoms of the disease not to develop in a subject that may be exposed to or predisposed to the disease but does not yet experience or display symptoms of the disease). The terms further include causing the clinical symptoms not to develop, for example in a subject at risk of suffering from such a disease or disorder, thereby substantially averting onset of the disease or disorder.

The term “effective amount” refers to an amount that is effective for the treatment of a condition or disorder by an intranasal administration of a compound or composition described herein. In some embodiments, an effective amount of any of the compositions or dosage forms described herein is the amount used to treat a neural disease, disorder or condition and/or to reduce one or more negative effects of a neural disease, disorder or condition comprising administering intranasally any of the compositions or dosage forms described herein to a subject in need thereof. In some embodiments, the condition or disorder that is treated with an effective amount of a compound or composition described herein is of the brain, spine and/or central nervous system.

The term “carrier” as used herein, refers to relatively nontoxic chemical compounds or agents that facilitate the incorporation of a compound into cells or tissues.

The term “axon” refers to projections of neurons that conduct signals to other cells through synapses. The term “axon growth” refers to the extension of the axon projection via the growth cone at the tip of the axon.

The term “neural disease” refers to diseases that compromise the cell viability of neurons. Neural diseases in which the etiology of said neural disease comprises formation of protein aggregates which are pathogenic to neurons provided that the protein aggregates are not related to the disease SBMA and are not intranuclear, include but are not limited to ALS, AD, Parkinson's Disease, multiple sclerosis, and prion diseases such as Kuru, Creutzfeltdt-Jakob disease, Fatal familial insomnia, and Gerstmann-Straussler-Scheinker syndrome. These neural diseases are also different from SBMA in that they do not contain polyglutamine repeats. Neural diseases can be recapitulated in vitro in tissue culture cells. For example, AD can be modeled in vitro by adding pre-aggregated 13-amyloid peptide to the cells. ALS can be modeled by depleting an ALS disease-related protein, TDP-43. Neural disease can also be modeled in vitro by creating protein aggregates through providing toxic stress to the cell. One way this can be achieved is by mixing dopamine with neurons such as neuroblastoma cells. These neural diseases can also be recapitulated in vivo in mouse models. A transgenic mouse that expresses a mutant Sodl protein has similar pathology to humans with ALS. Similarly, a transgenic mouse that overexpresses APP has similar pathology to humans with AD.

### Compounds:

### GGA

This invention relates to compounds and pharmaceutical compositions of isomers of geranylgeranyl acetone. In certain aspects, this invention relates to a synthetic 5-trans isomer compound of formula VI:

[chemical structure]

wherein VI is at least 80% in the 5E, 9E, 13E configuration. In some embodiments, the invention provides for a compound of formula VI wherein VI is at least 85%, or at least 90%, or at least 95%, or at least 96%, or at least 97%, or at least 98%, or at least 99%, or at least 99.5%, or at least 99.9% in the 5E, 9E, 13E configuration. In some embodiments the invention for the compound of formula VI does not contain any of the cis-isomer of GGA.

Another aspect of this invention relates to a synthetic 5-cis isomer compound of formula VII:

[chemical structure]

wherein VII is at least 75% in the 5Z, 9E, 13E configuration. In certain embodiments, the invention provides for a compound of formula VII wherein VII is at least 80% in the 5E, 9E, 13E configuration, or alternatively, at least 85%, or at least 90%, or at least 95%, or at least 96%, or at least 97%, or at least 98%, or at least 99%, or at least 99.5%, or at least 99.9% in the 5E, 9E, 13E configuration. In some embodiments of the invention, the compound of formula VII does not contain any of the trans-isomer of GGA.

The configuration of compounds can be determined by methods known to those skilled in the art such as chiroptical spectroscopy and nuclear magnetic resonance spectroscopy.

The data contained in the examples herewith demonstrate at low concentrations the trans-isomer of GGA is pharmacologically active and shows a dose-dependent relationship. In contrast, the cis-isomer of GGA does not demonstrate a dose dependent relationship and is deemed to be at best of minimal activity.

### GGA Derivatives

GGA derivatives useful in this invention include those described in PCT publication no. WO 2012/031028 and PCT application no. PCT/US2012/027147, each of which are incorporated herein by reference in its entirety. These and other GGA derivatives provided and/or utilized herein are structurally shown below.

In one aspect, the GGA derivative provided and/or utilized herein is of Formula I:

[chemical structure]

or a tautomer or pharmaceutically acceptable salt thereof, wherein
n¹ is 1 or 2;
each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;
each of R³, R⁴, and R⁵ independently are hydrogen or C₁-C₆ alkyl;

### Q¹ is —(C═O)—, —(C═S)—, or —S(O₂)—;

Q₂ is hydrogen, R⁶, —O—R⁶, —NR⁷R⁸, or is a chiral moiety;

### R⁶ is:

C₁-C₆ alkyl, optionally substituted with —CO₂H or an ester thereof, C₁-C₆ alkoxy, oxo, —OH, —CR═CR₂, —C≡CR, C₃-C₁₀ cycloalkyl, C₃-C₈ heterocyclyl, C₆-C₁₀ aryl, C₂-C₁₀ heteroaryl, wherein each R independently is hydrogen or C₁-C₆ alkyl;

CO—C₁-C₆ alkyl;

C₃-C₁₀ cycloalkyl;

C₃-C₈ heterocyclyl;

C₆-C₁₀ aryl; or

C₂-C₁₀ heteroaryl;

wherein each cycloalkyl, heterocyclyl, aryl, or heteroaryl is optionally substituted with 1-3 alkyl groups; —CF₃, 1-3 halo, preferably, chloro or fluoro, groups; 1-3 nitro groups; 1-3 C₁-C₆ alkoxy groups; —CO-phenyl; or —NR¹⁸R¹⁹, each R¹⁸ and R¹⁹ independently is hydrogen; C₁-C₁₀ alkyl, optionally substituted with —CO₂H or an ester thereof, C₁-C₆ alkoxy, oxo, —CR═CR₂, —C≡CR, C₃-C₁₀ preferably C₃-C₈ cycloalkyl, C₃-C₈ heterocyclyl, C₆-C₁₀ aryl, or C₂-C₁₀ heteroaryl, wherein each R independently is hydrogen or C₁-C₆ alkyl; C₃-C₁₀ cycloalkyl; C₃-C₈ heterocyclyl; C₆-C₁₀ aryl; or C₂-C₁₀ heteroaryl; wherein each cycloalkyl, heterocyclyl, aryl, or heteroaryl is optionally substituted with 1-3 alkyl groups, optionally substituted with 1-3 halo, preferably, fluoro, groups, where R¹⁸ and R¹⁹ together with the nitrogen atom they are attached to form a 5-7 membered heterocycle;

each R⁷ and R⁸ are independently hydrogen or defined as R⁶; and

[symbol] refers to a mixture of cis and trans isomers at the corresponding position wherein at least 80% and, preferably, no more than 95% of the compound of Formula (I) is present as a trans isomer.

In one embodiment, the GGA derivative provided and/or utilized is of Formula (I-A):

[chemical structure]

as a substantially pure trans isomer around the 2,3 double bond wherein, n¹,R¹-R⁵, Q¹, and Q² are defined as in Formula (I) above.

In another embodiment, n¹ is 1. In another embodiment, n¹ is 2.

In another embodiment, the GGA derivative provided and/or utilized is of Formula (I-B):

[chemical structure]

as a substantially pure trans isomer around the 2,3 double bond wherein, R¹-R⁵, Q¹, and Q² are defined as in Formula (I) above.

In another embodiment, the GGA derivative provided and/or utilized is of Formula I-C:

[chemical structure]

wherein Q¹ and Q² are defined as in Formula (I) above.

In another embodiment, the GGA derivative provided and/or utilized is of Formula (I-D), (I-E), or (I-F):

[chemical structure]

wherein R⁶-R⁸ are defined as in Formula (I) above.

In another embodiment, the GGA derivative provided and/or utilized is of Formula (I-G), (I-H), or (I-I):

[chemical structure]

as a substantially pure trans isomer around the 2,3 double bond wherein R⁶-R⁸ are defined as in Formula (I) above.

In a preferred embodiment, R⁶ is C₆-C₁₀ aryl, such as naphthyl. In another preferred embodiment, R⁶ is a heteroaryl, such as quinolinyl.

In another aspect, the GGA derivative provided and/or utilized in this invention is of Formula (II):

[chemical structure]

or a pharmaceutically acceptable salt thereof, wherein
m is 0 or 1;
n is 0, 1, or 2;
each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;
each of R³, R⁴, and R⁵ independently are hydrogen or C₁-C₆ alkyl;
Q₃ is —OH, —NR²²R²³—X—CO—NR²⁴R²⁵, —X—CS—NR²⁴R²⁵, or —X—SO₂—NR²⁴R²⁵;

### X is —O—, —S—, —NR²⁶—, or —CR²⁷R²⁸;

each R²² and R²³ independently is hydrogen; C₁-C₆ alkyl, optionally substituted with C₁-C₆ alkoxy; and C₃-C₁₀ cycloalkyl;
each R²⁴ and R²⁵ independently is hydrogen, C₁-C₆ alkyl, optionally substituted with —CO₂H or an ester thereof, C₁-C₆ alkoxy, oxo, —OH, —CR═CR₂, C₃-C₁₀ cycloalkyl, C₃-C₈ heterocyclyl, C₆-C₁₀ aryl, C₂-C₁₀ heteroaryl, wherein each R independently is hydrogen or C₁-C₆ alkyl;

C₃-C₁₀ cycloalkyl;

C₃-C₈ heterocyclyl;

C₆-C₁₀ aryl; or

C₂-C₁₀ heteroaryl;

wherein each cycloalkyl, heterocyclyl, aryl, or heteroaryl is optionally substituted with 1-3 alkyl groups; —CF₃, 1-3 halo, preferably, chloro or fluoro, groups; 1-3 nitro groups; 1-3 C₁-C₆ alkoxy groups; —CO-phenyl; or —NR¹⁸R¹⁹;
each R¹⁸ and R¹⁹ independently is hydrogen; C₁-C₆ alkyl, optionally substituted with —CO₂H or an ester thereof, C₁-C₆ alkoxy, oxo, —CR═CR₂, —C≡CR, C₃-C₁₀ preferably C₃-C₈ cycloalkyl, C₃-C₈ heterocyclyl, C₆-C₁₀ aryl, or C₂-C₁₀ heteroaryl, wherein each R independently is hydrogen or C₁-C₆ alkyl; C₃-C₁₀ cycloalkyl; C₃-C₈ heterocyclyl; C₆-C₁₀ aryl; or C₂-C₁₀ heteroaryl; wherein each cycloalkyl, heterocyclyl, aryl, or heteroaryl is optionally substituted with 1-3 alkyl groups, optionally substituted with 1-3 halo, preferably, fluoro, groups, where R¹⁸ and R¹⁹ together with the nitrogen atom they are attached to form a 5-7 membered heterocycle;
R²⁶ is hydrogen or together with R²⁴ or R²⁵ and the intervening atoms form a 5-7 membered heterocyclic ring optionally substituted with 1-3 C₁-C₆ alkyl groups; and
each R²⁷ and R²⁸ independently are hydrogen, C₁-C₆ alkyl, —COR⁸¹ or —CO₂R⁸¹, or R²⁷ together with R²⁴ or R²⁵ and the intervening atoms form a 5-7 membered heterocyclyl ring optionally substituted with 1-3 C₁-C₆ alkyl groups.

As used herein, the compound of Formula (II) includes optical isomers such as enantiomers and diastereomers. As also used herein, an ester refers preferably to a phenyl or a C₁-C₆ alkyl ester, which phenyl or alkyl group is optionally substituted with a amino group.

In one embodiment, Q₃ is —NR²²R²³—X—CO—NR²⁴R²⁵, X—CS—NR²⁴R²⁵, or —X—SO₂—NR²⁴R²⁵. In another embodiment, Q₃ is —X—CO—NR²⁴R²⁵, —X—CS—NR²⁴R²⁵, or —X—SO₂—NR²⁴R²⁵.

In another embodiment, Q₃ is —NR²²R²³. In another embodiment, Q₃ is —OH.

In one embodiment, the compound of Formula (II) is of formula:

[chemical structure]

wherein R¹, R², R³, R⁴, R⁵, and Q₃ are defined as in any aspect or embodiment herein.

In another embodiment, the GGA derivative provided and/or utilized is of formula:

[chemical structure]

wherein R¹, R², R⁴, R⁵, and Q₃ are defined as in any aspect and embodiment here.

In one embodiment, the compound of Formula (II) is of formula:

[chemical structure]

wherein R¹, R², R³, R⁴, R⁵, and Q₃ are defined as in any aspect or embodiment herein.

In another embodiment, the GGA derivative provided and/or utilized is of formula:

[chemical structure]

wherein R¹, R², R⁴, R⁵, m, n, X, R²⁴ and R²⁵ are defined as in any aspect and embodiment here.

In another embodiment, the GGA derivative provided and/or utilized is of formula:

[chemical structure]

wherein R¹, R², R⁴, R⁵, m, n, and R²⁴ are defined as in any aspect and embodiment here.

In another embodiment, the GGA derivative provided and/or utilized is of formula:

[chemical structure]

wherein R²⁴ is defined as in any aspect and embodiment here.

In another embodiment, the GGA derivative provided and/or utilized is of formula:

[chemical structure]

wherein R²⁴ is defined as in any aspect and embodiment here.

In another embodiment, the GGA derivative provided and/or utilized is of formula:

[chemical structure]

wherein R²⁴ is defined as in any aspect and embodiment here.

In another embodiment, the GGA derivative provided and/or utilized is of formula:

[chemical structure]

wherein R²⁴ is defined as in any aspect and embodiment here.

In another embodiment, the GGA derivative provided and/or utilized is of formula:

[chemical structure]

wherein R²⁴ and R²⁵ are defined as in any aspect and embodiment here.

In another embodiment, the GGA derivative provided and/or utilized is of formula:

[chemical structure]

wherein R²⁴ is defined as in any aspect and embodiment here.

In another embodiment, the GGA derivative provided and/or utilized is of formula:

[chemical structure]

wherein R²⁴ and R²⁵ are defined as in any aspect and embodiment here.

In one embodiment, m is 0. In another embodiment, m is 1.

In another embodiment, n is 0. In another embodiment, n is 1. In another embodiment, n is 2.

In another embodiment, m+n is 1. In another embodiment, m+n is 2. In another embodiment, m+n is 3.

In another embodiment, R¹ and R² are independently C₁-C₆ alkyl. In another embodiment, R¹ and R² independently are methyl, ethyl, or isopropyl.

In another embodiment, R¹ and R² together with the carbon atom they are attached to form a C₅-C₇ cycloalkyl ring optionally substituted with 1-3 C₁-C₆ alkyl groups. In another embodiment, R¹ and R² together with the carbon atom they are attached to form a ring that is:

[chemical structure]

In another embodiment, R³, R⁴, and R⁵ are independently C₁-C₆ alkyl. In another embodiment, one of R³, R⁴, and R⁵ are alkyl, and the rest are hydrogen. In another embodiment, two of R³, R⁴, and R⁵ are alkyl, and the rest are hydrogen. In another embodiment, R³, R⁴, and R⁵ are hydrogen. In another embodiment, R³, R⁴, and R⁵ are methyl.

In another embodiment, Q₃ is —X—CO—NR²⁴R²⁵. In another embodiment, Q₃ is —X—CS—NR²⁴R²⁵. In another embodiment, Q₃ is —X—SO₂—NR²⁴R²⁵. In another embodiment, Q₃ is —OCONHR²⁴, —OCONR²⁴R²⁵, —NHCONHR²⁴, NHCONR²⁴R²⁵, —OCSNHR²⁴, —OCSNR²⁴R²⁵, —NHCSNHR²⁴, or —NHCSNR²⁴R²⁵.

In another embodiment, X is —O—. In another embodiment, X is —NR²⁶—. In another embodiment, X is or —CR²⁷R²⁸.

In another embodiment, one of R²⁴ and R²⁵ is hydrogen. In another embodiment, one or both of R²⁴ and R²⁵ are C₁-C₆ alkyl. In another embodiment, one or both of R²⁴ and R²⁵ are C₁-C₆ alkyl, optionally substituted with an R²⁰ group, wherein R²⁰ is —CO₂H or an ester thereof, C₁-C₆ alkyl, C₃-C₁₀ cycloalkyl, C₃-C₈ heterocyclyl, C₆-C₁₀ aryl, or C₂-C₁₀ heteroaryl. In another embodiment, one or both of R²⁴ and R²⁵ are C₃-C₁₀ cycloalkyl. In another embodiment, one or both of R²⁴ and R²⁵ are C₃-C₁₀ cycloalkyl substituted with 1-3 alkyl groups. In another embodiment, one or both of R²⁴ and R²⁵ are C₃-C₈ heterocyclyl. In another embodiment, one or both of R²⁴ and R²⁵ are C₆-C₁₀ aryl. In another embodiment, one or both of R²⁴ and R²⁵ are C₂-C₁₀ heteroaryl. In another embodiment, R²⁴ and R²⁵ together with the nitrogen atom they are attached to form a 5-7 membered heterocycle.

In another embodiment, R²⁰ is —CO₂H or an ester thereof. In another embodiment, R²⁰ is C₁-C₆ alkyl. In another embodiment, R²⁰ is C₃-C₁₀ cycloalkyl. In another embodiment, R²⁰ is C₃-C₈ heterocyclyl. In another embodiment, R²⁰ is C₆-C₁₀ aryl. In another embodiment, R²⁰ is or C₂-C₁₀ heteroaryl.

In another embodiment, the GGA derivative provided and/or utilized is of formula (II):

[chemical structure]

or a pharmaceutically acceptable salt thereof, wherein
   m is 0 or 1;
   n is 0, 1, or 2;
   each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;
   each of R³, R⁴, and R⁵ independently are hydrogen or C₁-C₆ alkyl;
   Q₃ is —X—CO—NR²⁴R²⁵ or —X—SO₂—NR²⁴R²⁵;
   X is —O—, —NR²⁶—, or —CR²⁷R²⁸;
   R²⁶ is hydrogen or together with R²⁴ or R²⁵ and the intervening atoms form a 5-7 membered ring optionally substituted with 1-3 C₁-C₆ alkyl groups;
   each R²⁷ and R²⁸ independently are hydrogen, C₁-C₆ alkyl, —COR⁸¹ or —CO₂R⁸¹, or R²⁷ together with R²⁴ or R²⁵ and the intervening atoms form a 5-7 membered cycloalkyl or heterocyclyl ring optionally substituted with 1-3 C₁-C₆ alkyl groups;
   each R²⁴ and R²⁵ independently is
   hydrogen,
   C₁-C₆ alkyl, optionally substituted with —CO₂H or an ester thereof, C₃-C₁₀ preferably C₃-C₈ cycloalkyl, C₃-C₈ heterocyclyl, C₆-C₁₀ aryl, or C₂-C₁₀ heteroaryl,
   C₃-C₁₀ cycloalkyl,
   C₃-C₈ heterocyclyl,
   C₆-C₁₀ aryl, or
   C₂-C₁₀ heteroaryl,
wherein each cycloalkyl, heterocyclyl, aryl, or heteroaryl is optionally substituted with 1-3 C₁-C₆ alkyl groups, or R²⁴ and R²⁵ together with the nitrogen atom they are attached to form a 5-7 membered heterocycle.

In another embodiment, provided herein are compounds of formula:

[chemical structure]

In another aspect, the GGA derivative provided and/or utilized herein is of Formula III:

[chemical structure]

or a pharmaceutically acceptable salt of each thereof, wherein

m is 0 or 1;

n is 0, 1, or 2;

each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;

each of R³, R⁴, and R⁵ independently are hydrogen or C₁-C₆ alkyl;

Q₄ is selected from the group consisting of:

[chemical structure]

when X¹ is bonded via a single bond, X¹ is —O—, —NR³¹—, or —CR³²R³³—, and when X¹ is bonded via a double bond, X¹ is —CR³²—;

Y¹ is hydrogen, —OH or —O—R¹⁰, Y² is —OH, —OR¹¹ or —NHR¹², or Y¹ and Y² are joined to form an oxo group (═O), an imine group (═NR¹³), a oxime group (═N—OR¹⁴), or a substituted or unsubstituted vinylidene (═CR¹⁶R¹⁷);

R³⁰ is C₁-C₆ alkyl optionally substituted with 1-3 alkoxy or 1-5 halo group, C₂-C₆ alkenyl, C₂-C₆ alkynyl, C₃-C₁₀ cycloalkyl, C₆-C₁₀ aryl, C₃-C₈ heterocyclyl, or C₂-C₁₀ heteroaryl, wherein each cycloalkyl or heterocyclyl is optionally substituted with 1-3 C₁-C₆ alkyl groups, or wherein each aryl or heteroaryl is independently substituted with 1-3 C₁-C₆ alkyl or nitro groups, or R³⁰ is —NR³⁴R³⁵;

R³¹ is hydrogen or together with R³⁰ and the intervening atoms form a 5-7 membered ring optionally substituted with 1-3 C₁-C₆ alkyl groups;

each R³² and R³³ independently are hydrogen, C₁-C₆ alkyl, —COR⁸¹ or —CO₂R⁸¹, or R³² together with R³⁰ and the intervening atoms form a 5-7 membered cycloalkyl or heterocyclyl ring optionally substituted with oxo or 1-3 C₁-C₆ alkyl groups;

R¹⁰ is C₁-C₆ alkyl;

R¹¹ and R¹² are independently C₁-C₆ alkyl, C₃-C₁₀ cycloalkyl, —CO₂R¹⁵, or —CON(R¹⁵)₂, or R¹⁰ and R¹¹ together with the intervening carbon atom and oxygen atoms form a heterocycle optionally substituted with 1-3 C₁-C₆ alkyl groups;

R¹³ is C₁-C₆ alkyl or C₃-C₁₀ cycloalkyl optionally substituted with 1-3 C₁-C₆ alkyl groups;

R¹⁴ is hydrogen, C₃-C₈ heterocyclyl, or C₁-C₆ alkyl optionally substituted with a —CO₂H or an ester thereof or a C₆-C₁₀ aryl, C₂-C₆ alkenyl, C₂-C₆ alkynyl, C₃-C₁₀ cycloalkyl, or a C₃-C₈ heterocyclyl, wherein each cycloalkyl, heterocyclyl, or aryl, is optionally substituted with 1-3 alkyl groups;

each R¹⁵ independently are hydrogen, C₃-C₁₀ cycloalkyl, C₁-C₆ alkyl optionally substituted with 1-3 substituents selected from the group consisting of —CO₂H or an ester thereof, aryl, or C₃-C₈ heterocyclyl, or two R¹⁵ groups together with the nitrogen atom they are bonded to form a 5-7 membered heterocycle;

R¹⁶ is hydrogen or C₁-C₆ alkyl;

R¹⁷ is hydrogen, C₁-C₆ alkyl substituted with 1-3 hydroxy groups, —CHO, or is CO₂H or an ester thereof;

each R³⁴ and R³⁵ independently is hydrogen, C₁-C₆ alkyl, optionally substituted with —CO₂H or an ester thereof, C₃-C₁₀ cycloalkyl, C₃-C₈ heterocyclyl, C₆-C₁₀ aryl, or C₂-C₁₀ heteroaryl, or is C₃-C₁₀ cycloalkyl, C₃-C₈ heterocyclyl, C₆-C₁₀ aryl, or C₂-C₁₀ heteroaryl, wherein each cycloalkyl, heterocyclyl, aryl, or heteroaryl is optionally substituted with 1-3 alkyl groups, or R³⁴ and R³⁵ together with the nitrogen atom they are attached to form a 5-7 membered heterocycle; and

each R⁸¹ independently is C₁-C₆ alkyl.

In one embodiment, m is 0. In another embodiment, m is 1. In another embodiment, n is 0. In another embodiment, n is 1. In another embodiment, n is 2.

In one embodiment, the compound of Formula (III) is of formula:

[chemical structure]

wherein Q₄, R¹, R², R³, R⁴, R⁵, R³⁰, X¹, Y¹, and Y² are defined as in any aspect or embodiment herein.

In one embodiment, the GGA derivative provided and/or utilized is of formula:

[chemical structure]

wherein R¹, R², R³, R⁴, R⁵, R³⁰, X¹, Y¹, and Y² are defined as in any aspect and embodiment here.

In another embodiment, the GGA derivative provided and/or utilized is of formula:

[chemical structure]

wherein R¹, R², R³, R⁴, R⁵, R³⁰, X¹, and Y² are defined as in any aspect and embodiment herein.

In another embodiment, the GGA derivative provided and/or utilized is of formula:

[chemical structure]

wherein R¹, R², R³, R⁴, R⁵, R³⁰ and X¹ are defined as in any aspect and embodiment herein.

In another embodiment, the GGA derivative provided and/or utilized is of formula:

[chemical structure]

wherein R¹, R², R⁴, R⁵, and Q₄ are defined as in any aspect and embodiment herein.

In another embodiment, the GGA derivative provided and/or utilized is of formula:

[chemical structure]

wherein R¹, R², R⁴, R⁵, m, n, X¹, and R³⁰ are defined as in any aspect and embodiment here.

In another embodiment, the GGA derivative provided and/or utilized is of formula:

[chemical structure]

wherein R¹, R², R⁴, R⁵, m, n, and R³⁴ are defined as in any aspect and embodiment here.

In another embodiment, the GGA derivative provided and/or utilized is of formula:

[chemical structure]

wherein R¹, R², R⁴, R⁵, R³⁰, m, n, and R¹⁵ are defined as in any aspect and embodiment here.

In another embodiment, each R¹ and R² are C₁-C₆ alkyl. In another embodiment, each R¹ and R² are methyl, ethyl, or isopropyl. In another embodiment, R¹ and R² together with the carbon atom they are attached to form a 5-6 membered ring optionally substituted with 1-3 C₁-C₆ alkyl groups. In another embodiment, R¹ and R² together with the carbon atom they are attached to form a ring that is:

[chemical structure]

In another embodiment, R³, R⁴, and R⁵ are C₁-C₆ alkyl. In another embodiment, one of R³, R⁴, and R⁵ are alkyl, and the rest are hydrogen. In another embodiment, two of R³, R⁴, and R⁵ are alkyl, and the rest are hydrogen. In another embodiment, R³, R⁴, and R⁵ are hydrogen. In another embodiment, R³, R⁴, and R⁵ are methyl.

In another embodiment, X¹ is O. In another embodiment, X¹ is —NR³¹. In another embodiment, R³¹ is hydrogen. In another embodiment, R³¹ together with R³⁰ and the intervening atoms form a 5-7 membered ring optionally substituted with 1-3 C₁-C₆ alkyl groups. In another embodiment, X¹ is —CR³²R³³—. In another embodiment, X¹ is —CR³²—. In another embodiment, each R³² and R³³ independently are hydrogen, C₁-C₆ alkyl, —COR⁸¹, or —CO₂R⁸¹. In another embodiment, R³² is hydrogen, and R³³ is hydrogen, C₁-C₆ alkyl, —COR⁸¹, or —CO₂R⁸¹.

In another embodiment, R³³ is hydrogen. In another embodiment, R³³C₁-C₆ alkyl. In another embodiment, R³³ is methyl. In another embodiment, R³³ is —CO₂R⁸¹. In another embodiment, R³³ is —COR⁸¹.

In another embodiment, R³² together with R³⁰ and the intervening atoms form a 5-7 membered ring. In another embodiment, the moiety:

[chemical structure]

which is “Q₄,” has the structure:

[chemical structure]

wherein R³³ is hydrogen, C₁-C₆ alkyl, or —CO₂R⁸¹ and n is 1, 2, or 3. Within these embodiments, in certain embodiments, R³³ is hydrogen or C₁-C₆ alkyl. In one embodiment, R³³ is hydrogen. In another embodiment, R³³ is C₁-C₆ alkyl.

In another embodiment, R³⁰ is C₁-C₆ alkyl. In another embodiment, R³⁰ is methyl, ethyl, butyl, isopropyl, or tertiary butyl. In another embodiment, R³⁰ is C₁-C₆ alkyl substituted with 1-3 alkoxy or 1-5 halo group. In another embodiment, R³⁰ is alkyl substituted with an alkoxy group. In another embodiment, R³⁰ is alkyl substituted with 1-5, preferably, 1-3, halo, preferably fluoro, groups.

In another embodiment, R³⁰ is NR³⁴R³⁵. In a preferred embodiment, R³⁵ is H. In a preferred embodiment, R³⁴ is C₁-C₆ alkyl, optionally substituted with a group selected from the group consisting of —CO₂H or an ester thereof, C₃-C₁₀ cycloalkyl, C₃-C₈ heterocyclyl, C₆-C₁₀ aryl, or C₂-C₁₀ heteroaryl. In another preferred embodiment, R³⁴ is C₃-C₁₀ cycloalkyl, C₃-C₈ heterocyclyl, C₆-C₁₀ aryl, or C₂-C₁₀ heteroaryl. In a more preferred embodiment, R³⁴ is C₃-C₁₀ cycloalkyl.

In another embodiment, R³⁰ is C₂-C₆ alkenyl or C₂-C₆ alkynyl. In another embodiment, R³⁰ is C₃-C₁₀ cycloalkyl. In another embodiment, R³⁰ is C₃-C₁₀ cycloalkyl substituted with 1-3 C₁-C₆ alkyl groups. In another embodiment, R³⁰ is cyclopropyl, cyclobutyl, cyclopentyl, cyclohexyl, or adamentyl. In another embodiment, R³⁰ is C₆-C₁₀ aryl or C₂-C₁₀ heteroaryl. In another embodiment, R³⁰ is a 5-7 membered heteroaryl containing at least 1 oxygen atom. In another embodiment, R³⁰ is C₆-C₁₀ aryl, C₃-C₈ heterocyclyl, or C₂-C₁₀ heteroaryl, wherein each aryl, heterocyclyl, or heteroaryl is optionally substituted with 1-3 C₁-C₆ alkyl groups.

In another embodiment, Y² is —O—R¹¹. In another embodiment, Y¹ and Y² are joined to form ═NR¹³. In another embodiment, Y¹ and Y² are joined to form ═NOR¹⁴. In another embodiment, Y¹ and Y² are joined to form (═O). In another embodiment, Y¹ and Y² are joined to form ═CR¹⁶R¹⁷.

In another embodiment, Q₄ is —CR³³COR³⁰. In another embodiment, R³⁰ is C₁-C₆ alkyl optionally substituted with an alkoxy group. In another embodiment, R³⁰ is C₃-C₈ cycloalkyl. In another embodiment, R³³ is hydrogen. In another embodiment, R³³ is C₁-C₆ alkyl. In another embodiment, R³³ is CO₂R⁸¹. In another embodiment, R³³ is COR⁸¹.

In another embodiment, Q₄ is —CH₂—CH(O—CONHR¹⁵)—R³⁰. In another embodiment, R¹⁵ is C₃-C₈ cycloalkyl. In another embodiment, R¹⁵ is C₁-C₆ alkyl optionally substituted with 1-3 substituents selected from the group consisting of —CO₂H or an ester thereof, aryl, or C₃-C₈ heterocyclyl. In a preferred embodiment within these embodiments, R³⁰ is C₁-C₆ alkyl.

In another embodiment, Q₄ is —O—CO—NHR³⁴. within these embodiment, in another embodiment, R³⁴ is C₁-C₆ alkyl, optionally substituted with —CO₂H or an ester thereof, C₃-C₈ cycloalkyl, C₃-C₈ heterocyclyl, C₂-C₁₀ aryl, or C₂-C₁₀ heteroaryl. In yet another embodiment, R³⁴ is C₃-C₈ cycloalkyl, C₃-C₈ heterocyclyl, C₂-C₁₀ aryl, or C₂-C₁₀ heteroaryl.

In another embodiment, R¹⁴ is hydrogen. In another embodiment, R¹⁴ is C₁-C₆ alkyl optionally substituted with a —CO₂H or an ester thereof or a C₆-C₁₀ aryl optionally substituted with 1-3 alkyl groups. In another embodiment, R¹⁴ is C₂-C₆ alkenyl. In another embodiment, R¹⁴ is C₂-C₆ alkynyl In another embodiment, R¹⁴ is C₃-C₆ cycloalkyl optionally substituted with 1-3 alkyl groups. In another embodiment, R¹⁴ is C₃-C₈ heterocyclyl optionally substituted with 1-3 alkyl groups.

In another embodiment, preferably, R¹⁶ is hydrogen. In another embodiment, R¹⁷ is CO₂H or an ester thereof. In another embodiment, R¹⁷ is C₁-C₆ alkyl substituted with 1-3 hydroxy groups. In another embodiment, R¹⁷ is C₁-C₃ alkyl substituted with 1 hydroxy group. In another embodiment, R¹⁷ is —CH₂OH.

In another embodiment, R¹⁰ and R¹¹ together with the intervening carbon atom and oxygen atoms form a heterocycle of formula:

[chemical structure]

wherein q is 0 or 1, p is 0, 1, 2, or 3, and R³⁶ is C₁-C₆ alkyl.

In another embodiment, q is 1. In another embodiment, q is 2. In another embodiment, p is 0. In another embodiment, p is 1. In another embodiment, p is 2. In another embodiment, p is 3.

In one aspect, the GGA derivative provided and/or utilized herein is of Formula (IV):

[chemical structure]

or a tautomer thereof, or a pharmaceutically acceptable salt of each thereof, wherein
m is 0 or 1;
n is 0, 1, or 2;
each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;
each of R³, R⁴, and R⁵ independently are hydrogen or C₁-C₆ alkyl, or R⁵ and Q₅ together with the intervening carbon atoms form a 6 membered aryl ring, or a 5-8 membered cycloalkenyl ring, or a 5-14 membered heteroaryl or heterocycle, wherein each aryl, cycloalkenyl, heteroaryl, or heterocycle, ring is optionally substituted with 1-2 substituents selected from the group consisting of halo, hydroxy, oxo, —N(R⁴⁰)₂, and C₁-C₆ alkyl group;
Q₅ is —C(═O)H, —CO₂H or —CH═CHCO₂H, or a C₁-C₆ alkyl ester or acyl halide thereof, wherein the ester is optionally substituted with —CO-phenyl; a 6-10 membered aryl or a 5-14 membered heteroaryl or heterocycle containing up to 6 ring heteroatoms, wherein the heteroatom is selected from the group consisting of O, N, S, and oxidized forms of N and S, and further wherein the aryl, heteroaryl, or heterocyclyl ring is optionally substituted with 1-3 substituents selected from the group consisting of:

hydroxy, oxo, —N(R⁴⁰)₂, C₁-C₆ alkoxy group, and C₁-C₆ alkyl group,

wherein the alkyl group is optionally substituted with 1-3 substituents selected from hydroxy, NH₂, C₆-C₁₀ aryl, —CO₂H or an ester or an amide thereof,
a 5-9 membered heteroaryl containing up to 3 ring heteroatoms, wherein the heteroaryl is optionally substituted with 1-3 hydroxy, —N(R⁴⁰)₂, and C₁-C₆ alkyl group,
benzyl, and phenyl optionally substituted with 1-3 substituents selected from the group consisting of C₁-C₆ alkyl, C₁-C₆ alkoxy, hydroxy, and halo groups; and
   wherein each R⁴⁰ independently is hydrogen or C₁-C₆ alkyl.

As used herein, the compound of Formula (IV) includes tautomers and optical isomers such as enantiomers and diastereomers. As also used herein, an ester refers preferably to a phenyl or a C₁-C₆ alkyl ester, which phenyl or alkyl group is optionally substituted with a amino group. As used herein, an amide refers preferably to a moiety of formula —CON(R⁴⁰)₂, wherein R⁴⁰ is defined as above.

In some embodiment, Q₆ is selected from a group consisting of oxazole, oxadiazole, oxazoline, azalactone, imidazole, diazole, triazole, and thiazole, wherein each heteroaryl or heterocycle is optionally substituted as disclosed above.

In one embodiment, the GGA derivative provided and/or utilized is of formula IV-A:

[chemical structure]

In another embodiment, the GGA derivative provided and/or utilized is of formula IV-B:

[chemical structure]

wherein R¹, R², R⁴, R⁵, and Q₅ are defined as in any aspect and embodiment here.

In another embodiment, Q₅ is selected from the group consisting of:

[chemical structure]

wherein R¹¹ is C₁-C₆ alkyl, C₆-C₁₀ aryl, C₃-C₈ heteroaryl, C₃-C₈ heteroaryl, C₃-C₁₀ cycloalkyl, and the alkyl group is optionally substituted with 1-3 C₆-C₁₀ aryl, C₃-C₈ heteroaryl, C₃-C₈ heteroaryl, C₃-C₁₀ cycloalkyl groups, and the aryl, heteroaryl, heteroaryl, cycloalkyl groups are optionally substituted with 1-3 C₁-C₆ alkyl, C₁-C₆ alkoxy, halo, preferably chloro or fluoro, C₆-C₁₀ aryl, C₃-C₈ heteroaryl, C₃-C₈ heteroaryl, C₃-C₁₀ cycloalkyl group.

In another embodiment, Q₅ is phenyl, optionally substituted as described herein. In another embodiment, Q₅ is benzimidazole, benzindazole, and such other 5-6 fused 9-membered bicyclic heteroaryl or heterocycle. In another embodiment, Q₅ is quinoline, isoquinoline, and such other 6-6 fused 10 membered heteroaryl or heterocycle. In another embodiment, Q₅ is benzodiazepine or a derivative thereof, such as, a benzodiazepinone. Various benzodiazepine and derivatives thereof are well known to the skilled artisan.

In another embodiment, m is 0. In another embodiment, m is 1.

In another embodiment, n is 0. In another embodiment, n is 1. In another embodiment, n is 2.

In another embodiment, m+n is 1. In another embodiment, m+n is 2. In another embodiment, m+n is 3.

In another embodiment, R¹ and R² are independently C₁-C₆ alkyl. In another embodiment, R¹ and R² independently are methyl, ethyl, or isopropyl.

In another embodiment, R¹ and R² together with the carbon atom they are attached to form a C₅-C₇ cycloalkyl ring optionally substituted with 1-3 C₁-C₆ alkyl groups. In another embodiment, R¹ and R² together with the carbon atom they are attached to form a ring that is:

[chemical structure]

In another embodiment, R³, R⁴, and R⁵ are independently C₁-C₆ alkyl. In another embodiment, one of R³, R⁴, and R⁵ are alkyl, and the rest are hydrogen. In another embodiment, two of R³, R⁴, and R⁵ are alkyl, and the rest are hydrogen. In another embodiment, R³, R⁴, an R⁵ are hydrogen. In another embodiment, R³, R⁴, and R⁵ are methyl.

In another embodiment, this invention provides a compound selected from the group consisting of:

[chemical structure]

wherein R¹¹ is defined as above.

In another aspect, GGA derivatives provided and/or utilized herein are of formula (V):

[chemical structure]

or a pharmaceutically acceptable salt thereof, wherein
m is 0 or 1;
n is 0, 1, or 2;
each R¹ and R² independently are C₁-C₆ alkyl, or R¹ and R² together with the carbon atom they are attached to form a C₅-C₇ cycloalkyl ring optionally substituted with 1-3 C₁-C₆ alkyl groups;
each of R³, R⁴, and R⁵ independently is hydrogen or C₁-C₆ alkyl;
Q₆ is selected from the group consisting of:

[chemical structure]

when X² is bonded via a single bond, X² is —O—, —NR⁵²—, or —CR⁵³R⁵⁴—, and when X² is bonded via a double bond, X² is —CR⁵³—;
Y¹¹ is hydrogen, —OH or —OR⁵⁵;
Y²² is —OH, —OR⁵⁶, —NHR⁵⁷, or —O—CO—NR⁵⁸R⁵⁹, or Y¹¹ and Y²² are joined to form an oxo group (═O), an imine group (═NR⁶⁰), a oxime group (═N—OR⁶¹), or a substituted or unsubstituted vinylidene (═CR⁶³R⁶⁴);
R⁵¹ is C₁-C₆ alkyl, C₁-C₆ alkyl substituted with 1-3 alkoxy or 1-5 halo groups, C₂-C₆ alkenyl, C₂-C₆ alkynyl, C₃-C₁₀ cycloalkyl, C₃-C₈ heterocyclyl, C₆-C₁₀ aryl, C₂-C₁₀ heteroaryl, or —NR⁶⁵R⁶⁶, wherein each cycloalkyl or heterocyclyl is optionally substituted with 1-3 C₁-C₆ alkyl groups, and wherein each aryl or heteroaryl is optionally substituted independently with 1-3 nitro and C₁-C₆ alkyl groups;
R⁵² is hydrogen or together with R⁵¹ and the intervening atoms form a 5-7 membered ring optionally substituted with 1-3 C₁-C₆ alkyl groups;
each R⁵³ and R⁵⁴ independently are hydrogen, C₁-C₆ alkyl, —COR⁸¹, —CO₂R⁸¹, or —CONHR⁸², or R⁵³ together with R⁵¹ and the intervening atoms form a 5-7 membered cycloalkyl or heterocyclyl ring optionally substituted with 1-3 C₁-C₆ alkyl groups;
R⁵⁵ is C₁-C₆ alkyl;
each R⁵⁶ and R⁵⁷ independently are C₁-C₆ alkyl, C₃-C₁₀ cycloalkyl, —CO₂R⁶², or —CON(R⁶²)₂; or R⁵⁵ and R⁵⁶ together with the intervening carbon atom and oxygen atoms form a heterocycle optionally substituted with 1-3 C₁-C₆ alkyl groups;
R⁵⁸ is: C₃-C₁₀ cycloalkyl, C₁-C₆ alkyl optionally substituted with —OH, CO₂H or an ester thereof, or C₃-C₁₀ cycloalkyl,

[chemical structure]

R⁵⁹ is hydrogen or C₁-C₆ alkyl;
R⁶⁰ is C₁-C₆ alkyl or C₃-C₁₀ cycloalkyl optionally substituted with 1-3 C₁-C₆ alkyl groups, or is:

[chemical structure]

R⁶¹ is hydrogen, C₃-C₈ heterocyclyl, or C₁-C₆ alkyl optionally substituted with a —CO₂H or an ester thereof or a C₆-C₁₀ aryl, C₂-C₆ alkenyl, C₂-C₆ alkynyl, C₃-C₁₀ cycloalkyl, or a C₃-C₈ heterocyclyl, wherein each cycloalkyl, heterocyclyl, or aryl, is optionally substituted with 1-3 alkyl groups;
each R⁶² independently are hydrogen, C₃-C₁₀ cycloalkyl, C₁-C₆ alkyl optionally substituted with 1-3 substituents selected from the group consisting of —CO₂H or an ester thereof, aryl, C₃-C₈ heterocyclyl, or two R⁶² groups together with the nitrogen atom they are bonded to form a 5-7 membered heterocycle;
R⁶³ is hydrogen or C₁-C₆ alkyl;
R⁶⁴ is hydrogen, C₁-C₆ alkyl substituted with 1-3 hydroxy groups, —CHO, or is CO₂H or an ester thereof;
one or both of R⁶⁵ and R⁶⁶ independently are hydrogen, C₁-C₆ alkyl, optionally substituted with —CO₂H or an ester thereof, C₃-C₁₀ cycloalkyl, C₃-C₈ heterocyclyl, C₂-C₁₀ aryl, or C₂-C₁₀ heteroaryl, or is C₃-C₁₀ cycloalkyl, C₃-C₈ heterocyclyl, C₆-C₁₀aryl, or C₂-C₁₀ heteroaryl, wherein each cycloalkyl, heterocyclyl, aryl, or heteroaryl is optionally substituted with 1-3 alkyl groups, or R⁶⁵ and R⁶⁶ gether with the nitrogen atom they are bonded to form a 5-7 membered heterocycle, and if only one of R⁶⁵ and R⁶⁶ are defined as above, then the other one is

[chemical structure]

and
R⁸¹ is C₁-C₆ alkyl; and

R⁸² is:

[chemical structure]

provided that, when X² is bonded via a single bond, and R⁵³ or R⁵⁴ is not —CONHR⁸², Y¹¹ and Y²² are joined to form an imine group (═NR⁶⁰), and R⁶⁰ is:

[chemical structure]

or Y²² is —O—CO—NR⁵⁸R⁵⁹;
or provided that, when Q₆ is:

[chemical structure]

and R⁵³ is not —CONHR⁸², Y²² is —O—CO—NR⁵⁸R⁵⁹;
or provided that, when Q₆ is —O—CO—NR⁶⁵R⁶⁶, then at least one of R⁶⁵ and R⁶⁶ is:

[chemical structure]

In one embodiment, the GGA derivative provided and/or utilized are of formula:

[chemical structure]

In another aspect, the GGA derivatives useful according to this invention is selected from:

[chemical structure]

In one embodiment, the compounds provided herein excludes the compound of formula:

[chemical structure]

wherein L is 0, 1, 2, or 3, and R¹⁷ is CO₂H or an ester thereof, or is —CH₂OH, or is a C₁-C₆ alkyl ester of —CH₂OH.

In another embodiment, examples of compounds provided and/or utilized by this invention include certain compounds tabulated below. Compound ID numbers in Table 1 refer to synthetic schemes in Example 7.

TABLE 1

| Compound ID | Structure |
|---|---|
| 1 | [chemical structure] |
| 2a | [chemical structure] |
| 2b | [chemical structure] |
| 2c | [chemical structure] |
| 2d | [chemical structure] |
| 2e | [chemical structure] |
| 2f | [chemical structure] |
| 2g | [chemical structure] |
| 2h | [chemical structure] |
| 2i | [chemical structure] |
| 2j | [chemical structure] |
| 2k | [chemical structure] |
| 2l | [chemical structure] |
| 4a | [chemical structure] |
| 4b | [chemical structure] |
| 4c | [chemical structure] |
| 6a | [chemical structure] |
| 6b | [chemical structure] |
| 7a | [chemical structure] |
| 7b | [chemical structure] |
| 7c | [chemical structure] |
| 7d | [chemical structure] |
| 7e | [chemical structure] |
| 7f | [chemical structure] |
| 7g | [chemical structure] |
| 7h | [chemical structure] |
| 7i | [chemical structure] |
| 7j | [chemical structure] |
| 7k | [chemical structure] |
| 7l | [chemical structure] |
| 7m | [chemical structure] |
| 7n | [chemical structure] |
| 7o | [chemical structure] |
| 7p | [chemical structure] |
| 7q | [chemical structure] |
| 7r | [chemical structure] |
| 7s | [chemical structure] |
| 7t | [chemical structure] |
| 7u | [chemical structure] |
| 7v | [chemical structure] |
| 7w | [chemical structure] |
| 7x | [chemical structure] |
| 7y | [chemical structure] |
| 7z | [chemical structure] |
| 7aa | [chemical structure] |
| 8a | [chemical structure] |
| 8b | [chemical structure] |
| 8c | [chemical structure] |
| 8d | [chemical structure] |
| 8e | [chemical structure] |
| 8f | [chemical structure] |
| 7g | [chemical structure] |
| 8h | [chemical structure] |
| 8i | [chemical structure] |
| 8j | [chemical structure] |
| 8k | [chemical structure] |
| 8l | [chemical structure] |
| 8m | [chemical structure] |
| 8n | [chemical structure] |
| 8o | [chemical structure] |
| 9a | [chemical structure] |
| 9b | [chemical structure] |
| 9c | [chemical structure] |
| 9d | [chemical structure] |
| 9e | [chemical structure] |
| 9f | [chemical structure] |
| 9g | [chemical structure] |
| 9h | [chemical structure] |
| 9i | [chemical structure] |
| 9j | [chemical structure] |
| 9k | [chemical structure] |
| 10a | [chemical structure] |
| 10b | [chemical structure] |
| 10c | [chemical structure] |
| 10d | [chemical structure] |
| 10e | [chemical structure] |
| 10f | [chemical structure] |
| 10g | [chemical structure] |
| 10h | [chemical structure] |
| 10i | [chemical structure] |
| 10j | [chemical structure] |
| 10k | [chemical structure] |
| 10l | [chemical structure] |
| 10m | [chemical structure] |
| 12 | [chemical structure] |
| 14 | [chemical structure] |
| 15 | [chemical structure] |
| 16 | [chemical structure] |
| 17a | [chemical structure] |
| 17b | [chemical structure] |
| 17c | [chemical structure] |
| 17d | [chemical structure] |
| 17e | [chemical structure] |
| 19 | [chemical structure] |
| 20a | [chemical structure] |
| 20b | [chemical structure] |
| 20c | [chemical structure] |
| 20d | [chemical structure] |
| 20e | [chemical structure] |
| 20f | [chemical structure] |
| 20g | [chemical structure] |
| 20h | [chemical structure] |
| 20i | [chemical structure] |
| 20j | [chemical structure] |
| 22 | [chemical structure] |
| 23a | [chemical structure] |
| 23b | [chemical structure] |
| 23c | [chemical structure] |
| 23d | [chemical structure] |
| 23d | [chemical structure] |
| 23f | [chemical structure] |
| 23g | [chemical structure] |
| 24 | [chemical structure] |
| 25 | [chemical structure] |
| 27a | [chemical structure] |
| 27b | [chemical structure] |
| 27c | [chemical structure] |
| 27d | [chemical structure] |
| 27e | [chemical structure] |
| 27f | [chemical structure] |
| 27g | [chemical structure] |
| 29a | [chemical structure] |
| 29b | [chemical structure] |
| 29c | [chemical structure] |
| 29d | [chemical structure] |
| 29e | [chemical structure] |
| 29f | [chemical structure] |
| 31 | [chemical structure] |
| 32 | [chemical structure] |
| 35a | [chemical structure] |
| 35b | [chemical structure] |
| 35c | [chemical structure] |
| 35d | [chemical structure] |
| 37a | [chemical structure] |
| 37b | [chemical structure] |
| 37c | [chemical structure] |
| 37d | [chemical structure] |
| 38a | [chemical structure] |
| 38b | [chemical structure] |
| 39 | [chemical structure] |
| 40a | [chemical structure] |
| 40b | [chemical structure] |
| 41 | [chemical structure] |
| 42 | [chemical structure] |
| 43 | [chemical structure] |

In another embodiment, examples of compounds provided and/or utilized by this invention include certain compounds tabulated below.

TABLE 2

| Compound | |
|---|---|
| ID | Chemical Structure |
| 51 | [chemical structure] |
| 52 | [chemical structure] |
| 54 | [chemical structure] |
| 55 | [chemical structure] |
| 56 | [chemical structure] |
| 57 | [chemical structure] |
| 58 | [chemical structure] |
| 59 | [chemical structure] |
| 60 | [chemical structure] |
| 61 | [chemical structure] |
| 62 | [chemical structure] |
| 63 | [chemical structure] |
| 64 | [chemical structure] |
| 65 | [chemical structure] |
| 66 | [chemical structure] |
| 67 | [chemical structure] |
| 68 | [chemical structure] |
| 69 | [chemical structure] |
| 70 | [chemical structure] |
| 71 | [chemical structure] |
| 72 | [chemical structure] |
| 73 | [chemical structure] |
| 74 | [chemical structure] |
| 75 | [chemical structure] |
| 76 | [chemical structure] |
| 77 | [chemical structure] |
| 78 | [chemical structure] |
| 6979 | [chemical structure] |
| 80 | [chemical structure] |
| 81 | [chemical structure] |
| 82 | [chemical structure] |
| 83 | [chemical structure] |
| 84 | [chemical structure] |
| 85 | [chemical structure] |
| 86 | [chemical structure] |
| 87 | [chemical structure] |
| 88 | [chemical structure] |
| 89 | [chemical structure] |
| 90 | [chemical structure] |
| 91 | [chemical structure] |
| 92 | [chemical structure] |
| 93 | [chemical structure] |
| 94 | [chemical structure] |
| 95 | [chemical structure] |
| 96 | [chemical structure] |
| 97 | [chemical structure] |
| 98 | [chemical structure] |
| 99 | [chemical structure] |
| 100 | [chemical structure] |
| 101 | [chemical structure] |
| 102 | [chemical structure] |
| 103 | [chemical structure] |
| 104 | [chemical structure] |
| 105 | [chemical structure] |
| 106 | [chemical structure] |
| 107 | [chemical structure] |
| 108 | [chemical structure] |
| 109 | [chemical structure] |
| 110 | [chemical structure] |
| 111 | [chemical structure] |
| 112 | [chemical structure] |
| 113 | [chemical structure] |
| 114 | [chemical structure] |
| 115 | [chemical structure] |
| 116 | [chemical structure] |
| 117 | [chemical structure] |
| 118 | [chemical structure] |
| 119 | [chemical structure] |
| 120 | [chemical structure] |
| 121 | [chemical structure] |
| 122 | [chemical structure] |
| 123 | [chemical structure] |
| 124 | [chemical structure] |
| 125 | [chemical structure] |
| 126 | [chemical structure] |
| 127 | [chemical structure] |
| 128 | [chemical structure] |
| 129 | [chemical structure] |
| 130 | [chemical structure] |
| 131 | [chemical structure] |
| 132 | [chemical structure] |
| 133 | [chemical structure] |
| 134 | [chemical structure] |
| 135 | [chemical structure] |
| 136 | [chemical structure] |
| 137 | [chemical structure] |
| 138 | [chemical structure] |
| 139 | [chemical structure] |
| 140 | [chemical structure] |
| 141 | [chemical structure] |
| 142 | [chemical structure] |
| 143 | [chemical structure] |
| 144 | [chemical structure] |
| 145 | [chemical structure] |
| 146 | [chemical structure] |
| 147 | [chemical structure] |

Illustrative and nonlimiting anticancer agents and conjugates and their methods of synthesis are shown below. Illustrative and nonlimiting viral agents, such as Vidarabine, and conjugates and their methods of synthesis are also shown below.

### Geranylgeranyl (GG)-Alcohol/Campothecin Conjugate

[chemical structure]

### Carbonate Containing GG-Alcohol/Campothecin Conjugate

[chemical structure]

### Carbamate GG-Alcohol/5-FU Codrug or Carrier Conjugate

[chemical structure]

### Vidarabine Conjugate

[chemical structure]

Other antiviral drugs may be attached in similar fashion to the GG-alcohol or GG-acetone.

Illustrative and non-limiting examples of antibiotics useful in such compounds and certain nonlimiting points of attachment (shown by an “4”) of such antibiotics to GGA or a GGA derivative are shown below.

[chemical structure]

Illustrative and non-limiting examples of glaucoma drugs useful in such compounds and certain nonlimiting points of attachment (shown by an “4”) of such drugs to GGA or a GGA derivative are shown below.

[chemical structure]

### Synthesis of GGA Derivatives

Certain methods for making GGA or certain GGA derivatives provided and/or utilized herein are described in PCT publication no. WO 2012/031028 and PCT application no. PCT/US2012/027147, each of which are incorporated herein by reference in its entirety. Other GGA derivatives can be prepared by appropriate substitution of reagents and starting materials, as will be well known to the skilled artisan upon reading this disclosure.

The reactions are preferably carried out in a suitable inert solvent that will be apparent to the skilled artisan upon reading this disclosure, for a sufficient period of time to ensure substantial completion of the reaction as observed by thin layer chromatography, ¹H-NMR, etc. If needed to speed up the reaction, the reaction mixture can be heated, as is well known to the skilled artisan. The final and the intermediate compounds are purified, if necessary, by various art known methods such as crystallization, precipitation, column chromatography, and the likes, as will be apparent to the skilled artisan upon reading this disclosure.

The compounds provided and/or utilized in this invention are synthesized, e.g., from a compound of formula (III-A):

[chemical structure]

wherein n, R¹-R⁵ and [symbol] are defined as in Formula (I) above, following various well known methods upon substitution of reactants and/or altering reaction conditions as will be apparent to the skilled artisan upon reading this disclosure. The compound of Formula (III-A) is itself prepared by methods well known to a skilled artisan, for example, and without limitation, those described in PCT Pat. App. Pub. No. WO 2012/031028 and PCT Pat. App. No. PCT/US2012/027147 (each supra). An illustrative and non-limiting method for synthesizing a compound of Formula (III-A), where n is 1, is schematically shown below.

[chemical structure]

[chemical structure]

Starting compound (iii), which is synthesized from compound (I) by adding isoprene derivatives as described here, is alkylated with a beta keto ester (iv), in the presence of a base such as an alkoxide, to provide the corresponding beta-ketoester (v). Compound (v) upon alkaline hydrolysis followed by decarboxylation provides ketone (vi). Keto compound (vi) is converted, following a Wittig Horner reaction with compound (vii), to the conjugated ester (viii). Compound (viii) is reduced, for example with LiAlH₄, to provide alcohol (ix).

As will be apparent to the skilled artisan, a compound of Formula (III), where n is 2, is synthesized by repeating the reaction sequence of alkylation with a beta-keto ester, hydrolysis, decarboxylation, Wittig-Horner olefination, and LiAlH₄ reduction.

Certain illustrative and non-limiting synthesis of compounds provided and/or utilized in this invention are schematically shown below. Compounds where Q¹ is or —SO₂— are synthesized by substituting the carbonyl group of the reactants employed, as will be apparent to the skilled artisan.

R⁶ in the schemes below may also correspond to R³⁰ and R⁵¹ as defined herein. R⁷ in the schemes below may also correspond to R²⁶, R³¹ and R⁵² as defined herein. R⁸ in the schemes below may also correspond to R²⁷, R³² and R⁵³ as defined herein. R⁹ in the schemes below may also correspond to R²⁸, R³³ and R⁵⁴ as defined herein. R¹³ in the schemes below may also correspond to R⁵⁸ as defined herein. R¹⁴ in the schemes below may also correspond to R⁵⁹ as defined herein. R¹⁵ in the schemes below may also correspond to R⁶⁰ as defined herein. R¹⁸ in the schemes below may also correspond to R²⁴, R³⁴ and R⁶³ as defined herein. R¹⁹ in the schemes below may also correspond to R²⁵, R³⁵ and R⁶⁴ as defined herein. L is a leaving group as known to one of ordinary skill in the art.

[chemical structure]

As shown above, R^{E} is alkyl.

Compound (ix) with alcohol functionality is an intermediate useful for preparing the compounds provided and/or utilized in this invention. Compound (x), where L is an RsSO₂— group is made by reacting compound (ix) with R^{s}SO₂Cl in the presence of a base. The transformation of compound (iii) to compound (x) illustrates methods of adding isoprene derivatives to a compound, which methods are suitable to make compound (iii) from compound (I). Intermediate (ix) containing various R¹-R⁵ substituents are prepared according to this scheme as exemplified herein below. The transformation of compound (iii) to compound (x) illustrates methods of adding isoprene derivatives to a compound, which methods are suitable to make compound (iii) from compound (I).

The intermediates prepared above are converted to the compounds provided and/or utilized in this invention as schematically illustrated below:

[chemical structure]

As used herein, for example, and without limitation, m is 0 or 1 and R¹-R⁵ are as defined herein, and are preferably alkyl, or more preferably methyl. Intermediate (ixa), prepared according to the scheme herein above, is converted to amino intermediate (ixb) via the corresponding bromide. Intermediates (ixa) and (ixb) are converted to the compounds provided and/or utilized in this invention by reacting with suitable isocyanates or carbamoyl chlorides, which are prepared by art known methods. The thiocarbamates and thioureas of this invention are prepared according to the methods described above and replacing the isocyanates or the carbamoyl chlorides with isothiocyanates (R¹⁸—N═C═S) or thiocarbamoyl chlorides (R¹⁸—NH—C(═S)Cl or R¹⁸R¹⁹N—C(═S)Cl). These and other compounds provided and/or utilized in this invention are also prepared by art known methods, which may require optional modifications as will be apparent to the skilled artisan upon reading this disclosure. Intermediates for synthesizing compounds provided and/or utilized in this invention containing various R¹-R⁵ substituents are illustrated in the examples section and/or are well known to the skilled artisan.

Certain GGA derivatives provided and/or utilized herein are synthesized as schematically shown below.

[chemical structure]

Certain compounds provided and/or utilized herein are obtained by reacting compound (x) with the anion Q(−), which can be generated by reacting the compound QH with a base. Suitable nonlimiting examples of bases include hydroxide, hydride, amides, alkoxides, and the like. Various compounds provided and/or utilized in this invention, wherein the carbonyl group is converted to an imine, a hydrazone, an alkoxyimine, an enolcarbamate, a ketal, and the like, are prepared following well known methods.

Other methods for making the compounds provided and/or utilized in this invention are schematically illustrated below:

[chemical structure]

The metallation is performed, by reacting the ketone with a base such as dimsyl anion, a hindered amide base such as diisopropylamide, or hexamethyldisilazide, along with the corresponding metal cation, M. The amino carbonyl chloride or the isocyanate is prepared, for example, by reacting the amine (R¹⁴)₂NH with phosgene or an equivalent reagent well known to the skilled artisan.

[chemical structure]

The beta keto ester is hydrolyzed while ensuring that the reaction conditions do not lead to decarboxylation. The acid is activated with various acid activating agent well known to the skilled artisan such as carbonyl diimodazole, or O-Benzotriazole-N,N,N,N-tetramethyl-uronium-hexafluoro-phosphate (HBTU) and reacted with the amine.

[chemical structure]

Various other compounds provided and/or utilized in this invention are prepared from the compounds made in the scheme above based on art known methods.

[chemical structure]

As shown above, R^{E} is alkyl.

The intermediates prepared above are converted to the compounds provided and/or utilized in this invention as schematically illustrated below:

[chemical structure]

Compound (viii) is hydrolyzed to the carboxylic acid (x), which is then converted to the acid chloride (xi). Compound (xi) is reacted with a suitable nucleophile such as a hydrazide, a hydroxylamine, an amino alcohol, or an amino acid, and the intermediate dehydrated to provide a compound of Formula (IV). Alternatively, the allylic alcohol (ix) is oxidized to the aldehyde (xi), which is then reacted with a cyanohydrin or cyanotosylmethane to provide further compounds provided and/or utilized in this invention.

GGA derivatives provided and/or utilized in this invention can also be synthesized employing art known methods and those disclosed here by alkene-aryl, alkene-heteroaryl, or alkene-alkene couplings such as Heck, Stille, or Suzuki coupling. Such methods can use (vi) to prepare intermediate (xii) that can undergo Heck, Stille, or Suzuki coupling under conditions well known to the skilled artisan to provide compounds provided and/or utilized in this invention.

[chemical structure]

Higher and lower isoprenyl homologs of intermediates (x), (xi), and (xii), which are prepared following the methods disclosed here, can be similarly employed to prepare other compounds provided and/or utilized in this invention.

Compounds provided and/or utilized in this invention are also prepared as shown below

[chemical structure]

L is a leaving group and Q₅ are as defined herein, Ar is a preferably an aryl group such as phenyl, the base employed is an alkoxide such as tertiarybutoxide, a hydride, or an alkyl lithium such as n-butyl lithium. Methods of carrying out the steps shown above are well known to the skilled artisan, as are conditions, reagents, solvents, and/or additives useful for performing the reactions and obtaining the compound of Formula (IV) in the desired stereochemistry.

Other methods for making the compounds provided and/or utilized in this invention are schematically illustrated below:

[chemical structure]

The metallation is performed, by reacting the ketone with a base such as dimsyl anion, a hindered amide base such as diisopropylamide, or hexamethyldisilazide, along with the corresponding metal cation, M. The amino carbonyl chloride or the isocyanate is prepared, for example, by reacting the amine R¹³R¹⁴NH with phosgene or an equivalent reagent well known to the skilled artisan.

[chemical structure]

The beta keto ester is hydrolyzed while ensuring that the reaction conditions do not lead to decarboxylation. The acid is activated with various acid activating agent well known to the skilled artisan such as carbonyl diimodazole, or O-Benzotriazole-N,N,N′,N′-tetramethyl-uronium-hexafluoro-phosphate (HBTU) and reacted with the amine. Certain other methods of preparing the conjugates are shown below.

[chemical structure]

As shown above, R is a memantine or a riluzole residue.
Polyprenyl amine-GGA derivatives can be prepared by reductive amination employing the appropriate polyprenyl aldehyde, a primary or secondary amine and a borohydride reducing agent, as is well known to the skilled artisan. The reaction can be carried out in THF or diethyl ether, optionally in presence of a protic acid, preferably a mild protic acid catalyst.

Illustrative and nonlimiting methods of making antibiotic and glaucoma drug conjugates of GGA and derivatives thereof are schematically shown below and/or can be adapted by the skilled artisan based on this disclosure. See, also, Expert Opinion on Therapeutic Patents, Prodrug strategies in nasal drug delivery, 2002, Vol. 12, No. 3, Pages 331-340.

### Ciprofloxacin Conjugate

[chemical structure]

### Betaxolol Conjugate

[chemical structure]

[chemical structure]

### Intranasal Formulations

In some aspects of the invention, a composition suitable for intranasal administration is provided for treatment of a neural disease, disorder or condition or for reducing the negative effects of a neural disease, disorder or condition, where the composition includes GGA, preferably all trans GGA, or a GGA derivative as described herein, or a pharmaceutically acceptable salt thereof, and at least one pharmaceutically acceptable excipient for introducing GGA and/or derivatives thereof via the intranasal route into a subject. The intranasal dosage form may be prepared using methods that are standard in the art (see e.g., Remington's Pharmaceutical Sciences, 16^{th} ed., A. Oslo editor, Easton Pa. 1980). The concentration of the excipient is one that can readily be determined to be effective by those skilled in the art, and can vary depending on the particular excipient used. The total concentration of the excipients in the solution can be from about 0.001% to about 90% or from about 0.001% to about 10%.

Currently, intranasal administration has not gained wide acceptance. For example, all therapeutic agents cannot be effectively administered by the intranasal route. At present, the molecules which have proved suitable for this route of administration are still very few and consist essentially of only small peptide or hormone molecules (such as calcitonin, cerulean, .beta.-endorphin, glucagon, horseradish peroxidase, B-interferon, oxytocin and insulin) in special formulations. The ability of drug molecules to be absorbed by the nasal mucous membranes is utterly unpredictable, as is the ability of intranasal formulations to avoid irritation of the mucous nasal membranes. Mucous membrane irritation caused by the drug and/or excipient is the most common reason for which intranasal administration has not gained wider acceptance.

The compositions according to the invention include GGA or a derivative thereof in quantities ranging from 1-55, 5-50, 10-40, or 20-30 mg/kg/day, diluted in excipients such as humectants, isotoning agents, antioxidants, buffers and preservatives. A calcium chelating agent is also preferably included.

The invention makes it possible to have single-dose dosage forms, which ensure application of an optimum quantity of GGA or a derivative thereof. The intranasal formulations of the invention contain concentrations of GGA or a derivative thereof ranging from 0.1 to 20%, preferably about 5-10% weight/volume. Selection of the particular excipients depends on the desired formulation dosage form, i.e., on whether a solution to be used in drops or as a spray (aerosol) is desired or a suspension, ointment or gel to be applied in the nasal cavity are desired.

Vehicles useful in the compositions according to the invention comprise solvent systems containing ethyl alcohol, isopropyl alcohol, propylene glycol, polyethylene glycol, mixtures thereof or mixtures of one or more of the foregoing with water.

Suitable vehicles for the formulations according to the invention include aqueous suspensions or emulsions containing an appropriate isotoning agent selected among those commonly used in pharmaceutics. Substances used for this purpose are, for instance, sodium chloride and glucose. The quantity of isotoning agent should impart to the vehicle (taking into account the osmotic effect of the active ingredient), an osmotic pressure similar to that of biological fluids, i.e. generally from about 150 to about 850 milliOsmoles (mOsm) preferably from about 270 to about 330 mOsm.

Nasal mucous membranes are also capable of tolerating slightly hypertonic solutions. Should a suspension or gel be prepared instead of a solution, appropriate oily or gel vehicles may be used or one or more polymeric materials may be included, which desirably should be capable of conferring bioadhesive characteristics to the vehicle.

Several polymers may be used for the preparation of a gel; nonlimiting examples include hydroxypropyl cellulose (KLUCEL®), hydroxypropyl methyl cellulose (METHOCEL®), hydroxyethyl cellulose (NATROSOL®), sodium carboxymethyl cellulose (BLANOSE®), acrylic polymers (CARBOPOL®, POLYCARBOPHIL®), gum xanthan, gum tragacanth, alginates and agar-agar.

Some of them, such as sodium carboxymethyl cellulose and acrylic polymers, have marked bioadhesive properties and are preferred if bioadhesiveness is desired.

Other formulations suitable for intranasal administration of GGA or a derivative thereof can be obtained by adding to the aqueous vehicle polymers capable of changing the rheologic behavior of the composition in relation to the temperature. These polymers make it possible to obtain low viscosity solutions at room temperature, which can be applied for instance by nasal spray and which increase in viscosity at body temperature, yielding a viscous fluid which ensures a better and longer contact with the nasal mucous membrane. Polymers of this class include without limitation polyoxyethylene-polyoxypropylene block copolymers (POLOXAMER®).

In some embodiments, the formulation is a small particle liposome or lipid complex aerosol formulation. Methods of preparing such formulation are within the skill of the skilled artisan. See, for example, U.S. Pat. No. 6,090,407.

In some embodiments, a pharmaceutically acceptable buffer is present to stabilize a pH range of about 4 to about 8; preferably about 5.5 to 7.5. Suitable non-limiting buffers include tris(tromethamine) buffer, phosphate buffer, etc.

Further excipients include chemical enhancers such as intranasal absorption promoters. These include chelating agents, fatty acids, bile acid salts and other surfactants, fusidic acid, lysophosphatides, cyclic peptide antibiotics, preservatives, carboxylic acids (ascorbic acid, amino acids), glycyrrhetinic acid, o-acylcarnitine. Preferred promoters are diisopropyladipate, POE (9) lauryl alcohol, sodium glycocholate and lysophosphatidyl-choline which proved to be particularly active. Finally, the new compositions according to the invention preferably contain preservatives which ensure the microbiological stability of the active ingredient. Suitable preservatives include without limitation, methyl paraoxybenzoate, propyl paraoxybenzoate, sodium benzoate, benzyl alcohol, benzalkonium chloride and chlorobutanol.

The liquid formulations of GGA or a derivative thereof, preferably in the form of solutions, may be administered from a nasal spray devise of this invention comprising GGA or a GGA derivative, in the form of drops or spray, using atomizers equipped with a mechanical valve and possibly including a propellant of a type commercially available, such as butane, N₂, Ar, CO₂, nitrous oxide, propane, dimethyl ether, chlorofluorocarbons (e.g. FREON) etc. Vehicles suitable for spray administration are water, alcohol, glycol and propylene glycol, used alone or in a mixture of two or more. In some embodiments, this invention provides mutidose nasal spray devices. In other embodiments, this invention provides unit dose nasal spray devices.

Generally, illustrative and non-limiting formulations can contain the following ingredients and amounts (weight/volme):

[Ingredient Broad Range (%) Preferred Range (%)] Na₂EDTA 0.001-1 0.05-0.1 Nipagin 0.01-2 0.05-0.25 POE (9) Lauryl alcohol 0.1-10 1-10 NaCMC (Blanose 7 m8 sfd) 0.1-5 0.3-3 Carbopol 940 0.05-2 0.1-1.5 Glycerol 1-99 Sodium glycocholate 0.05-5 0.1-1

It will be appreciated by those of ordinary skill that ingredients such as sodium carboxymethyl cellulose and Carbopol exist in many types differing in viscosity. Their amounts are to be adjusted accordingly. Different adjustments to each formulation may also be necessary including omission of some optional ingredients and addition of others. It is thus not possible to give an all-encompassing amount range for each ingredient, but the optimization of each preparation according to the invention is within the skill of the art.

An alternative for the intranasal administration of compositions including GGA or a derivative thereof comprises a suspension of finely micronized active ingredient (generally from 1 to 200 micrometers, preferably from 5 to 100 micrometers) in a propellant or in an oily vehicle or in another vehicle in which the drug is not soluble. The vehicle is mixed or emulsified with the propellant. Vehicles suitable for this alternative are, for instance, vegetable and mineral oils and triglyceride mixtures. Appropriate surfactants, suspending agents and diluents suitable for use in pharmaceutics are added to these vehicles. Surfactants include without limitation sorbitan sesquioleate, sorbitanmonooleate, sorbitan trioleate (amount: between about 0.25 and about 1%); suspending agents include without limitation isopropylmyristate (amount: between about 0.5 and about 1%) and colloidal silica (amount: between about 0.1 and about 0.5%); and diluents include without limitation zinc stearate (about 0.6 to about 1%).

In certain preferred embodiments of this invention, there is provided a pharmaceutical composition comprising GGA or a GGA derivative and α-tocopherol. A related embodiment provides for a pharmaceutical composition comprising GGA or a GGA derivative, α-tocopherol, and hydroxypropyl cellulose. In another embodiment, there is provided a pharmaceutical composition comprising GGA or a GGA derivative, α-tocopherol, and optionally gum arabic. In a further embodiment, there is a pharmaceutical composition comprising GGA or a GGA derivative, and gum arabic. In a related embodiment, there is provided GGA or a GGA derivative, gum arabic and hydroxypropyl cellulose.

When α-tocopherol is used alone or in combination with other excipients, the concentration by weight can be from about 0.001% to about 1% or from about 0.001% to about 0.005%, or from about 0.005% to about 0.01%, or from about 0.01% to about 0.015%, or from about 0.015% to about 0.03%, or from about 0.03% to about 0.05%, or from about 0.05% to about 0.07%, or from about 0.07% to about 0.1%, or from about 0.1% to about 0.15%, or from about 0.15% to about 0.3%, or from about 0.3% to about 0.5%, or from about 0.5% to about 1% by weight. In some embodiments, the concentration of α-tocopherol is about 0.001% by weight, or alternatively about 0.005%, or about 0.008%, or about 0.01%, or about 0.02%, or about 0.03%, or about 0.04%, or about 0.05% by weight.

When hydroxypropyl cellulose is used alone or in combination with other excipients, the concentration by weight can be from about 0.1% to about 30% or from about 1% to about 20%, or from about 1% to about 5%, or from about 1% to about 10%, or from about 2% to about 4%, or from about 5% to about 10%, or from about 10% to about 15%, or from about 15% to about 20%, or from about 20% to about 25%, or from about 25% to about 30% by weight. In some embodiments, the concentration of hydroxypropyl cellulose is about 1% by weight, or alternatively about 2%, or about 3%, or about 4%, or about 5%, or about 6%, or about 7%, or about 8%, or about 10%, or about 15% by weight.

When gum arabic is used alone or in combination with other excipients, the concentration by weight can be from about 0.5% to about 50% or from about 1% to about 20%, or from about 1% to about 10%, or from about 3% to about 6%, or from about 5% to about 10%, or from about 4% to about 6% by weight. In some embodiments, the concentration of hydroxypropyl cellulose is about 1% by weight, or alternatively about 2%, or about 3%, or about 4%, or about 5%, or about 6%, or about 7%, or about 8%, or about 10%, or about 15% by weight.

In certain embodiments, the concentration of GGA or a GGA derivative in the pharmaceutical composition is about 5% by weight, or alternatively, about 10%, or about 20%, or about 1%, or about 2%, or about 3%, or about 4%, or about 6%, or about 7%, or about 8%, or about 9%, or about 11%, or about 12%, or about 14%, or about 16%, or about 18% by weight.

The intranasal compositions comprising GGA or a GGA derivative of this invention maybe used alone or in combination with other compounds or compositions. When administered with another agent, the co-administration can be in any manner in which the pharmacological effects of both are manifest in the patient at the same time. Thus, co-administration does not require that a single pharmaceutical composition, the same dosage form, or even the same route of administration be used for administration of both the compound of this invention and the other agent or that the two agents be administered at precisely the same time. However, co-administration will be accomplished most conveniently by the same dosage form and the same route of administration, at substantially the same time. Obviously, such administration most advantageously proceeds by delivering both active ingredients simultaneously in a novel pharmaceutical composition in accordance with the present invention.

In some embodiments, a compound of this invention can be used as an adjunct to conventional drug therapy of the conditions described herein.

### Methods of Treatment

Some embodiments provided herein describe a method of treating a neural disease via an intranasal administration of GGA or a derivative thereof. In some instances, neural diseases are characterized by neuroinflammation. Examples of such neural diseases include, but are not limited to, amyotrophic lateral sclerosis (ALS), Alzheimer's disease, Parkinson's disease, multiple sclerosis, prion diseases such as Kuru, Creutzfeltdt-Jakob disease, fatal familial insomnia, Gerstmann-Straussler-Scheinker syndrome, and damage to the spinal cord. Also provided herein in some embodiments is a method of treating visual disorders such as optic neuropathy, glaucoma, degeneration of optic nerves, age-related macular degeneration (AMD) and ophthalmoplegia. Some embodiments described herein provide a pharmaceutical formulation for preventing neural death during epileptic seizures. Any pharmaceutical formulation and/or compounds described above are useful in the methods described herein.

Provided herein, in some embodiments, are methods for using effective amounts of GGA or a derivative thereof preferably having the (5E,9E,13E) configuration or the, optionally with at least one pharmaceutically acceptable excipient for inhibiting neural death and/or increasing neural activity. In some embodiments, GGA is the trans-GGA or the synthetic trans-GGA. For example, and without limitation, methods provided here in describe impeding the progression of neural diseases or injury using GGA or a derivative thereof.

In one aspect, methods for increasing the axon growth of neurons by contacting said neurons with the pharmaceutical compositions are provided herein. In some cases, neural diseases result in an impairment of signaling between neurons. In some cases, this impairment is due in part to a reduction in the growth of axonal projections. In some embodiments, contacting neurons with GGA or a derivative thereof enhances axonal growth. In some embodiments, GGA or a derivative thereof restores axonal grown in neurons afflicted with a neural disease. In a related embodiment, the pre-contacted neurons exhibit a reduction in the axon growth ability.

One embodiment provided herein describes a method for inhibiting the cell death of neurons susceptible to neuronal cell death, which method comprises contacting said neurons with the pharmaceutical compositions provided herein. Neurons susceptible to neuronal cell death include those that have the characteristics of a neural disease and/or those that have undergone injury or toxic stress. One method of creating toxic stress to a cell is by mixing dopamine with neurons such as neuroblastoma cells. Another source of toxic stress is oxidative stress. Oxidative stress can occur from neuronal disease or injury. It is contemplated that contacting neurons with GGA or a derivative thereof will inhibit their death as measured by a MTT assay or other techniques commonly known to one skilled in the art.

In another aspect, there are methods for increasing the neurite growth of neurons by contacting said neurons with the pharmaceutical compositions provided herein. The term “neurite” refers to both axons and dendrites. Neural diseases can result in an impairment of signaling between neurons. In some cases, this impairment is due in part to a reduction in the growth of axonal and/or dendritic projections. It is contemplated that contacting neurons with GGA or a derivative thereof will enhance neurite growth. It is further contemplated that GGA or a derivative thereof will restore neurite grown in neurons afflicted with a neural disease. In a related embodiment, the pre-contacted neurons exhibit a reduction in the neurite growth ability.

One embodiment of this invention is directed to a method for increasing the expression and/or release of one or more neurotransmitters from a neuron by contacting said neurons with the pharmaceutical compositions provided herein. It is contemplated that contacting neurons with an effective amount of GGA and/or derivatives thereof will increase the expression level of one or more neurotransmitters. It is also contemplated that contacting neurons with GGA or a derivative thereof will increase the release of one or more neurotransmitters from neurons. The release of one or more neurotransmitters refers to the exocytotic process by which secretory vesicles containing one or more neurotransmitters are fused to cell membrane, which directs the neurotransmitters out of the neuron. It is contemplated that the increase in the expression and/or release of neurotransmitters will lead to enhanced signaling in neurons, in which levels of expression or release of neurotransmitters are otherwise reduced due to the disease. The increase in their expression and release can be measured by molecular techniques commonly known to one skilled in the art.

One embodiment of this invention is directed to a method for inducing synapse formation of a neuron by contacting said neurons with the pharmaceutical compositions provided herein. A synapse is a junction between two neurons. Synapses are essential to neural function and permit transmission of signals from one neuron to the next. Thus, an increase in the neural synapses will lead to an increase in the signaling between two or more neurons. It is contemplated that contacting the neurons with an effective amount GGA or a derivative thereof, via intranasal administration, will increase synapse formation in neurons that otherwise experience reduced synapse formation as a result of neural disease.

Another embodiment of this invention is directed to a method for increasing electrical excitability of a neuron by contacting said neurons with the pharmaceutical compositions provided herein. Electrical excitation is one mode of communication among two or more neurons. It is contemplated that contacting neurons with an effective amount of GGA or a derivative thereof, via intranasal administration, will increase the electrical excitability of neurons in which electrical excitability and other modes of neural communication are otherwise impaired due to neural disease. Electrical excitability can be measured by electrophysiological methods commonly known to one skilled in the art.

In each of the three previous paragraphs above, the intranasal administration of GGA or a derivative thereof enhances communication between neurons and accordingly provides for a method of inhibiting the loss of cognitive abilities in a mammal that is at risk of dementia or suffering from incipient or partial dementia while retaining some cognitive skills. Incipient or partial dementia in a mammal is one in which the mammal still exhibits some cognitive skills, but the skills are being lost and/or diminished over time. Method comprises administering via intranasal the route to said patient an effective amount of GGA or a derivative thereof.

In another embodiment, this invention is directed to a method for inhibiting the death of neurons due to formation of or further formation of pathogenic protein aggregates between, outside or inside neurons, wherein said method comprises contacting said neurons at risk of developing said pathogenic protein aggregates with the pharmaceutical compositions provided herein, provided that said pathogenic protein aggregates are not related to SBMA. In one embodiment of this invention, the pathogenic protein aggregates form between or outside of the neurons. In another embodiment of this invention, the pathogenic protein aggregates form inside said neurons. In one embodiment of this invention, the pathogenic protein aggregates are a result of toxic stress to the cell. One method of creating toxic stress to a cell is by mixing dopamine with neurons such as neuroblastoma cells. It is contemplated that contacting neurons with an effective amount of GGA or a derivative thereof, via intranasal administration, will inhibit their death as measured by a MTT assay or other techniques commonly known to one skilled in the art.

Another embodiment of the invention is directed to a method for protecting neurons from pathogenic extracellular protein aggregates which method comprises contacting said neurons and/or said pathogenic protein aggregates with the pharmaceutical compositions provided herein. In one embodiment of this invention, contacting said neurons and/or said pathogenic protein aggregates with the pharmaceutical compositions provided herein. Without being limited to any theory, it is contemplated that contacting the neurons and/or the pathogenic protein aggregates with GGA or a derivative thereof, via intranasal administration, will solubilize at least a portion of the pathogenic protein aggregates residing between, outside, or inside of the cells. It is further contemplated that contacting the neurons and/or the pathogenic protein aggregates with GGA or a derivative thereof, via intranasal administration, will alter the pathogenic protein aggregates in such a way that they are non-pathogenic. A non-pathogenic form of the protein aggregate is one that does not contribute to the death or loss of functionality of the neuron. There are many assays known to one skilled in the art for measuring the protection of neurons either in cell culture or in a mammal. One example is a measure of increased cell viability by a MTT assay. Another example is by immunostaining neurons in vitro or in vivo for cell death-indicating molecules such as, for example, caspases or propidium iodide.

In yet another embodiment of the invention is directed to a method for protecting neurons from pathogenic intracellular protein aggregates which method comprises contacting said neurons with the pharmaceutical compositions provided herein provided that said protein aggregation is not related to SBMA. This method is not intended to inhibit or reduce negative effects of neural diseases in which the pathogenic protein aggregates are intranuclear or diseases in which the protein aggregation is related to SBMA. SBMA is a disease caused by pathogenic androgen receptor protein accumulation. It is distinct from the neural diseases mentioned in this application since the pathogenic protein aggregates of SBMA contain polyglutamines and are formed intranuclearly. It is also distinct from the neural diseases described in this application because the protein aggregates are formed from androgen receptor protein accumulation. It is contemplated that contacting neurons via intranasal administration with an effective amount of GGA or a derivative thereof will alter the pathogenic protein aggregate into a non-pathogenic form.

One embodiment of the invention is directed to a method of modulating the activity of G proteins in neurons which method comprises contacting said neurons with the pharmaceutical compositions provided herein. It is contemplated that contacting neurons via intranasal administration with an effective amount of GGA or a derivative thereof will alter the sub-cellular localization, thus changing the activities of the G protein in the cell. In one embodiment of the invention, contacting neurons via intranasal administration with an effective amount of GGA and/or derivatives thereof will enhance the activity of G proteins in neurons. It is contemplated that contacting neurons via intranasal administration with an effective amount of GGA or a derivative thereof will increase the expression level of G proteins. It is also contemplated that contacting neurons via intranasal administration with an effective amount of GGA or a derivative thereof will enhance the activity of G proteins by changing their sub-cellular localization to the cell membranes where they must be to exert their biological activities.

One embodiment of the invention is directed to a method of modulating or enhancing the activity of G proteins in neurons at risk of death which method comprises contacting said neurons with the pharmaceutical compositions provided herein. Neurons may be at risk of death as a result of genetic changes related to ALS. One such genetic mutation is a depletion of the TDP-43 protein. It is contemplated that neurons with depleted TDP-43 or other genetic mutations associated with ALS will have an increase or change in the activity of G proteins after being contacted via intranasal administration with an effective amount of GGA or a derivative thereof. It is further contemplated that an effective amount of GGA or a derivative thereof will result in an increase in the activity of G proteins in these cells by changing their sub-cellular localization to the cell membranes where they must be to exert their biological activities.

Another embodiment of the invention is directed to a method for inhibiting the neurotoxicity of β-amyloid peptide by contacting the β-amyloid peptide with the pharmaceutical compositions provided herein. In one embodiment of the invention the β-amyloid peptide is between or outside of neurons. In yet another embodiment of the invention, the β-amyloid peptide is part of the β-amyloid plaque. It is contemplated that contacting neurons via intranasal administration with an effective amount of GGA or a derivative thereof will result in solubilizing at least a portion of the β-amyloid peptide, thus decreasing its neurotoxicity. It is further contemplated that an effective amount of GGA or a derivative thereof will decrease the toxicity of the β-amyloid peptide by altering it in such a way that it is no longer toxic to the cell. It is also believed that an effective amount of GGA and/or derivatives thereof will induce the expression of heat shock proteins (HSPs) in the neurons. It is also contemplated that HSPs will be induced in support cells such as glial cells. The induced heat shock proteins in the neurons or glial cells may be transmitted extracellularly and act to dissolve extracellular protein aggregates. Cell viability can be measured by standard assays known to those skilled in the art. One such example of an assay to measure cell viability is a MTT assay. Another example is a MTS assay. The modulation of protein aggregation can be visualized by immunostaining or histological staining techniques commonly known to one skilled in the art.

One embodiment of the invention is directed to a method for inhibiting neural death and increasing neural activity in a mammal suffering from neural diseases, wherein the etiology of said neural diseases comprises formation of protein aggregates which are pathogenic to neurons, and which method comprises administering to said mammal the pharmaceutical compositions provided herein. This method is not intended to inhibit neural death and increase neural activity in neural diseases in which the pathogenic protein aggregates are intranuclear or diseases in which the protein aggregation is related to SBMA.

Neural diseases such as AD and ALS disease have the common characteristic of protein aggregates either inside neural cells in cytoplasm or in the extracellular space between two or more neural cells. This invention relates to a method for using, via intranasal administration, an effective amount of GGA or a derivative thereof to inhibit the formation of the protein aggregates or alter the pathogenic protein aggregates into a non-pathogenic form. It is contemplated that this will attenuate some of the symptoms associated with these neural diseases.

In one embodiment the mammal is a human afflicted with a neural disease. In one embodiment of this invention, the negative effect of the neural disease being inhibited or reduced is ALS. ALS is characterized by a loss of functionality of motor neurons. This results in the inability to control muscle movements. ALS is a neurodegenerative disease that does not typically show intranuclear protein aggregates. It is contemplated that an effective amount of GGA and/or derivatives thereof will prevent or inhibit the formation of extracellular or intracellular protein aggregates that are cytoplasm, not intranuclear and not related to SBMA. It is also contemplated that an effective amount of GGA or a derivative thereof will alter the pathogenic protein aggregates into a form that is non-pathogenic. Methods for diagnosing ALS are commonly known to those skilled in the art. Additionally, there are numerous patents that describe methods for diagnosing ALS. These include U.S. Pat. No. 5,851,783 and U.S. Pat. No. 7,356,521 both of which are incorporated herein by reference in their entirety.

In one embodiment of the invention the negative effect of the neural disease being inhibited or reduced is AD. AD is a neurodegenerative disease that does not typically show intranuclear protein aggregates. It is contemplated the intranasal administration of an effective amount of GGA or a derivative thereof will prevent or inhibit the formation of extracellular or intracellular protein aggregates. It is also contemplated that the intranasal administration of an effective amount of GGA or a derivative thereof will alter the pathogenic protein aggregates into a form that is non-pathogenic. Methods for diagnosing AD are commonly known to those skilled in the art. Additionally, there are numerous patents that describe methods for diagnosing AD. These include U.S. Pat. No. 6,130,048 and U.S. Pat. No. 6,391,553 both of which are incorporated herein by reference in their entirety.

In another embodiment, the mammal is a laboratory research mammal such as a mouse. In one embodiment of this invention, the neural disease is ALS. One such mouse model for ALS is a transgenic mouse with a Sodl mutant gene. It is contemplated that the intranasal administration of an effective amount of GGA or a derivative thereof will enhance the motor skills and body weights when administered to a mouse with a mutant Sodl gene. It is further contemplated that the intranasal administration of an effective amount of GGA or a derivative thereof to this mouse will increase the survival rate of Sodl mutant mice. Motor skills can be measured by standard techniques known to one skilled in the art. In yet another embodiment of this invention, the neural disease is AD. One example of a transgenic mouse model for AD is a mouse that overexpresses the APP (Amyloid beta Precursor Protein). It is contemplated that the intranasal administration of an effective amount of GGA or a derivative thereof to a transgenic AD mouse will improve the learning and memory skills of said mouse. It is further contemplated that the intranasal administration of an effective amount of GGA or a derivative thereof will decrease the amount and/or size of β-amyloid peptide and/or plaque found inside, between, or outside of neurons. The β-amyloid peptide or plaque can be visualized in histology sections by immunostaining or other staining techniques.

In one embodiment of the invention the intranasal administration of an effective amount of GGA or a derivative thereof to a mammal alters the pathogenic protein aggregate present into a non-pathogenic form. In another embodiment of the invention, the intranasal administration of an effective amount of GGA or a derivative thereof to a mammal will prevent pathogenic protein aggregates from forming.

Another aspect of this invention relates to a method for reducing seizures in a mammal in need thereof, which method comprises administering the pharmaceutical compositions provided herein, thereby reducing seizures. The reduction of seizures refers to reducing the occurrence and/or severity of seizures. In one embodiment, the seizure is epileptic seizure. In another embodiment, the methods of this invention prevent neural death during epileptic seizures. The severity of the seizure can be measured by one skilled in the art.

In some embodiments, an intranasal formulation of GGA or a derivative thereof described herein exerts cytoprotective effects on a variety of organs, e.g., the brain and heart. (See, for example Tanito M, et al., J Neurosci 2005; 25:2396-404; Fujiki M, et al., J Neurotrauma 2006; 23:1164-78; Yasuda H, et al., Brain Res 2005; 1032:176-82; Ooie T, et al., Circulation 2001; 20; 104:1837-43; and Suzuki S, et al., Kidney Int 2005; 67:2210-20).

Method of treating bacterial infections, viral infections, or cancers of the eye, brain, and spinal chord, and the nerves in the brain, eye, and the spinal chord are well known in the art and can be appropriately adapted for practicing the methods of this invention upon reading this disclosure by the skilled artisan.

### EXAMPLES

The following examples of formulations for the intranasal administration of GGA or a GGA derivative serve to illustrate the invention without limiting its scope.

### Example 1

| Composition | % | For 10 liters | |
|---|---|---|---|
| GGA or a GGA derivative | 0.1-20% | 10-2,000 | g |
| EDTA disodium (chelating agent) | 0.01-0.1 | 1-10 | g |
| NIPAGIN (preservative) ** | 0.1-0.5 | 10-50 | g |
| Purified water | 100 | 10 | L |

### Methylparaoxybenzoate (Nipagin): BDH Chemical LTD, Poole, Dorset, UK

### Method of Preparation

In a suitable vessel equipped with mixer and heating sleeve, introduce about 9 liters of purified water and heat to a temperature of 80° C. Dissolve Nipagin and EDTA disodium. Stir the solution constantly to complete dissolution of the components. Cool the obtained solution to room temperature.
Dissolve or suspend GGA or a GGA derivative by stirring. Bring to volume with water. The isotonicity of this composition can be adjusted, if needed, by the addition e.g., of 0.3% NaCl or 2.03% of glucose.

All abbreviations for scientific terms used herein have their ordinary scientific meaning as known to the skilled artisan.

1. An intranasal composition, the composition comprising an effective amount of geranylgeranyl acetone (GGA) or a GGA derivative, or a pharmaceutically acceptable salt thereof, and a pharmaceutically acceptable excipient.

2. The composition of claim 1, wherein the GGA or a GGA derivative exists at least 80%, or at least 90%, or at least 95%, or at least 99% in the trans isomer.

3. The composition of claim 1 comprising 0.1-20% (weight/volume) of GGA or a GGA derivative, or a pharmaceutically acceptable salt thereof.

4. The composition of claim 1 comprising 5-10% (weight/volume) of GGA or a GGA derivative, or a pharmaceutically acceptable salt thereof.

5. The composition of claim 1 in the form of a solution, suspension or emulsion.

6. The composition of claim 1 wherein said excipient comprises a bioadhesive and/or an intranasal absorption promoter.

7. The composition of claim 6, wherein said intranasal absorption promoter is selected from the group consisting of a chelating agent.

8. A method comprising administering intranasally an effective amount of a composition of claim 1 to a subject in need thereof.

9. A method for treating a neural disease, disorder or condition and/or reducing one or more negative effects of a neural disease, disorder or condition comprising administering intranasally an effective amount of a composition of claim 1 to a subject in need thereof.
